(19) United States
(12) Patent Application Publication
(10) Pub. No.: US 2021/0257045 A1
(43) Pub. Date: Aug. 19, 2021

(54) METHOD FOR VERIFYING CULTIVATION DEVICE PERFORMANCE

(71) Applicant: Hoffmann-La Roche Inc., Little Falls, NJ (US)

(72) Inventors: Tobias GROSSKOPF, Penzberg (DE); Oliver POPP, Penzberg (DE); Tobias WALLOCHA, Penzberg (DE)

(73) Assignee: Hoffmann-La Roche Inc., Little Falls, NJ (US)

(21) Appl. No.: 17/186,816

(22) Filed: Feb. 26, 2021

Related U.S. Application Data
(63) Continuation of application No. PCT/EP2019/072538, filed on Aug. 23, 2019.

(30) Foreign Application Priority Data
Aug. 27, 2018 (EP) 18190942.5

(51) Int. Cl. G16B 5/00; G16B 50/10

(52) U.S. Cl. CPC G16B 5/00; G16B 50/10

(57) ABSTRACT

Herein is reported a method for determining if process data acquired during the cultivation of a mammalian or bacterial cell is affected by a problem comprising the steps of (i) fitting the process data acquired during the cultivation of a mammalian or bacterial cell clone expressing a recombinant, heterologous polypeptide in a metabolic model generated for the same mammalian or bacterial cell expressing the same recombinant, heterologous polypeptide, and (ii) determining that the cultivation is affected by a problem if the modeled fit shows an offset with respect to the raw data of more than 10%, or the modeled fit has a chi² value determined by a Pearson's chi-squared test of more than 5.

### CROSS-REFERENCE TO RELATED APPLICATIONS

This application is a Continuation of International Application No. PCT/EP2019/072538, filed Aug. 23, 2019, which claims benefit of priority to European Patent Application No. 18190942.5 filed Aug. 27, 2018. The contents of each of the foregoing applications are incorporated herein by reference in its entirety.

The current invention is in the field of cell cultivation, more precisely in the field of high-throughput cell cultivation. Herein are reported methods for determining if a cell cultivation is affected by a problem. The alignment or/and consistency control of experimentally determined data exploits amongst other things in silico metabolic modelling. By using metabolic flux analysis through a cellular model the consistency of in vitro data can be checked based on the fit between model and experiment.

### BACKGROUND OF THE INVENTION

Modem biotherapeutics meet a growing demand in the treatment of complex multifactorial diseases like cancer, diabetes, or rheumatoid arthritis. Most biotherapeutics are produced in established mammalian cell lines like, for example, Chinese Hamster Ovary (CHO) cells or well characterized bacterial strains like Escherichia coli (E. coli).

Cell line development and process development has traditionally been time-consuming and cumbersome for cell based bioprocesses due to, amongst other things, the need for gene amplification and clone selection. This conflicts with the need to rapidly provide sufficient material for many drug candidates undergoing pre-clinical and clinical evaluation.

The speeding up of cell line development for the production of biotherapeutics has generated a strong momentum for the development of supporting in silico methods for time and labor intensive in vitro and in vivo methods.

In-depth characterization of high-producer cell lines and bioprocesses is vital to ensure profound selection of the appropriate clones for robust and consistent production of biotherapeutics in high quantity and quality for pre-clinical and clinical applications. This requires applying appropriate methods during bioprocess development to enable meaningful characterization of cell clones and processes.

Recent progress in online process monitoring with process analytical technologies (PAT) and an increased focus on critical product quality attributes in industrial cell culture have greatly increased the breadth and amount of process data available today.

For extracting information from such enriched process data and to predict bioprocess performance, multivariate data analysis founded on statistical models assumes a predominant position among the methods applied at present (Pais et al., Curr. Opin. Biotechnol. 30C (2014) 161-167; Schaub et al., in: Hu W S, Zeng A-P, editors. Genomics and systems biology of mammalian cell culture. Berlin Heidelberg: Springer. 2012, 133-163. http://link.springer.com/chapter/10.1007/10_2010_98). In parallel, mechanistic metabolic models have been developed for several mammalian cell lines (Dietmair et al., Biotechnol. Bioeng. 109 (2012) 1404-1414; Nolan and Lee, Metab. Eng. 13 (2011) 108-124; Provost et al., Bioprocess Biosyst. Eng. 29 (2006) 349-366; Selvarasu et al., Mol. Biosyst. 6 (2010) 152-161; Sheikh et al., Biotechnol. Prog. 21 (2005) 112-121). Such models are attractive because they can capitalize on newly available genomic information (Birzele et al., Nucl. Acids Res. 38 (2010) 3999-4010; Brinkrolf et al., Nat. Biotechnol. 31 (2013) 694-695; Lewis et al., Nat. Biotechnol. 31 (2013) 759-765) and on quantitative metabolite measurements to enable a comprehensive assessment of the intracellular state simply based on extracellular data alone. In this way, they also interface nicely with scale-down fermentations systems like micro bioreactors in process development and clone selection (Bareither and Pollard, Biotechnol. Prog. 27 (2011) 2-14; Hsu et al., Cytotechnol. 64 (2012) 667-678). These systems enjoy increasing acceptance because they offer more predictive clone characterization as process conditions can be kept closer to controlled process scenarios like at larger scales (Porter et al., Biotechnol. Prog. 26 (2010) 446-1454 and 1455-1464; Rameez et al., Biotechnol. Prog. 30 (2014) 718-727).

Charaniya, S., et al. (J. Biotechnol. 147 (2010) 186-197) disclosed mining manufacturing data for discovery of high productivity process characteristics. Therein a kernel-based approach combined with a maximum margin-based support vector regression algorithm was used to integrate all the process parameters and develop predictive models for a key cell culture performance parameter. The model was also used to identify and rank process parameters according to their relevance in predicting process outcome.

Popp, O., et al. (Biotechnol. Bioeng. 113 (2016) 2005-2019) disclosed a hybrid approach for supporting comprehensive characterization of metabolic clone performance. This approach combined metabolite profiling with multivariate data analysis and fluxomics to enable a data-driven mechanistic analysis of key metabolic traits associated with desired cell phenotypes. The authors have applied the methodology to quantify and compare metabolic performance in a set of 10 recombinant CHO—K1 producer clones and a host cell line and were able to derive an extended set of clone performance criteria that not only captured growth and product formation, but also incorporated information on intracellular clone physiology and on metabolic changes during the process. Using these criteria allowed a quantitative clone ranking and allowed to identify metabolic differences between high-producing CHO—K1 clones yielding comparably high product titers.

WO 2011/140093 disclosed a method of assessing the severity of nonalcoholic fatty liver disease, nonalcoholic steatohepatitis, and/or liver fibrosis in a subject which includes obtaining a bodily sample from a subject and determining a level of the at least one oxidized fatty acid product in the sample when compared to the sample of a healthy individual.

WO 2011/136515 disclosed that only recently, genome-scale technologies enabled a system-level analysis to elucidate the complex biomolecular basis of protein production in mammalian cells promising an increased process understanding and the deduction of knowledge-based approaches for further process optimization. The document described a method for a rational cell culturing process using such a knowledge-based approach.

Paul, W., et al. (https://dc.engconfintl.org/ccexvi/161/) disclosed new approach in metabolic/process modeling and results. They have found that hybrid metabolic models enable a new approach for intracellular metabolic flux calculation and prediction of the metabolic status of the cell of tomorrow. These models enable metabolic driven process control. Artificial neural network succeeded metabolic flux estimation values with high confidence and low root mean squared error on average (RMSE) ˜20% (POC).

Current metabolic model based methods rely on manually inserted data into developed models. Corrupt data input can occur and harm the whole model output.

When performed at all, outlier detection methods rely on erratic data structure but not on biological relevance and cross-validation of data.

### SUMMARY OF THE INVENTION

One aim of the current invention was to provide methods for the identification or determination of cell cultures affected by a problem, i.e. the alignment and/or consistency control of experimentally determined data, using in silico modelling and metabolic flux analysis. By determining the goodness of fit between the model and the experimental data cultivations affected by a problem can be identified. The problem can either be a technical problem or a biological problem. A technical problem is based on a failure in the hardware used for performing the cultivation. A biological problem is based on the cell as such, e.g. resulting from bacterial or fungal contamination of the cultivation. Preferably the problem is a technical problem associated with the hardware, i.e. probes, vessels, electronic, devices, analytics etc., used for performing and/or monitoring the cultivation.

Thus, herein are provided methods for verifying cultivation performance by determining the goodness of fit (GoF) of experimental data to a (established) metabolic model.

All methods according to the current invention comprise either the following first set of steps:
fitting the process data acquired during the cultivation of a cell clone expressing a recombinant, heterologous polypeptide, preferably an antibody, using a metabolic model generated for the same cell expressing the same recombinant, heterologous polypeptide, and
determining that the cultivation is affected by a problem when the obtained fit shows an offset with respect to the raw data of more than 10%;

or the following second set of steps:
receiving process data of a cultivation of a cell clone, wherein the cell clone produces a polypeptide, preferably an antibody, heterologous to said cell,
fitting the data using a metabolic model established for said cell and characterizing the fit by a chi² (also written as χ²) value determined by a statistical correlation method, preferably a Pearson's chi-squared test, and
identifying the process data to be affected by a problem if the chi² value is more than 5.

### DETAILED DESCRIPTION OF EMBODIMENTS OF THE INVENTION

Definitions

The term “flux analysis” as used herein denotes the mathematical examination of biochemical stoichiometric reactions and pathways.

The term “flux balance analysis” short “FBA” as used herein denotes the optimization of biochemical stoichiometric models by means of linear algebra in order to maximize or minimize a given objective function.

The term “constraint flux analysis” as used herein denotes flux analysis, wherein the maximum and minimum allowed flux values for each reaction in the metabolic model are constrained to not surpass a specified value.

The term “genome-scale” as used herein denotes the exhaustive mapping of genetic capabilities of an organism onto biochemical stoichiometric reactions. Genome-scale models are derived from the sequenced genomic information of an organism and further curated by literature information and through experimental validation.

The term “in-process control” as used herein denotes methods and approaches for assessment of continuous (e.g. values within minutes) or discrete (e.g. one value every day) values, amounts and levels of physical cultivation parameters and cell phenotype and metabotype parameters needed for controlling, analyzing and interpreting a cultivation process. The parameters can be generated either on-line, at-line, or off-line for this purpose.

The term “process data” as used herein denotes the sum of on-line and off-line acquired temporal process parameter values including (calculated) outcome variables, such as rates. The “process data” is acquired in a time-dependent manner and archived, i.e. stored. The term “process data” as used herein includes at least the variables viability; viable cell density; viable cell volume; consumption and production rates of nutrients, such as e.g. glucose, phosphate, amino acids, fatty acids, as well as metabolites, such as e.g. lactate, ammonia; product; process-associated parameters, such as e.g. the physical parameters temperature, dissolved oxygen concentration, pH, aeration rate, reactor mass, added corrections fluids, and/or added feed. As the process parameters forming the process data are analytical values these are prone to technical problems. These technical problems relate amongst other things e.g. to the sampling, to the used analytical devices or, if humans are involved, to human errors.

The term “mammalian cell clone” as used herein denotes a mammalian cell that has been transfected with a nucleic acid encoding a secreted, heterologous polypeptide and that is expressing said secreted, heterologous polypeptide.

The term “metabolic flux analysis” short “MFA” as used herein denotes the mapping of measured fluxes over biochemical reactions onto a biochemical stoichiometric model and subsequent minimization of the total error within the model.

The term “metabolic network (re)construction” as used herein denotes the combination of activities that lead to the construction of a metabolic reaction network. Besides the collection of the biochemical pathway information, the curation and validation of the metabolic network are required to acquire a functional metabolic network reconstruction.

The term “multivariate data analysis” as used herein denotes the observation and analysis of multiple parameters in conjunction with respect to a statistical or mathematical analysis.

The term “network model” as used herein denotes the mathematical representation of an organism's biochemical reaction network.

The term “parental mammalian cell” as used herein denotes a mammalian cell prior to the transfection with a nucleic acid encoding a secreted, heterologous polypeptide.

The term “validation of in-process-recorded data” as used herein denotes checking data generated within a fermentation system by measurement for plausibility.

The term “statistical correlation method” as used herein denotes a statistical method by which it can be shown i) whether, and ii) how strongly pairs of variables are related to each other.

The term “Pearson's chi-squared test” as used herein denotes a method for calculating whether an observed frequency distribution differs from a theoretical distribution. It's a correlation coefficient, which is the covariance of two variables divided by the product of their standard deviations. Its result is a measure of the linear correlation between two variables X and Y. It has a value between +1 and −1, where 1 is total positive linear correlation, 0 is no linear correlation, and −1 is total negative linear correlation.

Model Generation

It is expressly pointed out that the following description of model generation is provided simply to provide written description of methods useful for carrying out the current invention. This is done to exemplify the current invention and not to limit it. A multitude of different methods and approaches for model building are known to a person skilled in the art and can be applied likewise in the method according to the invention.

The methods according to the current invention can be performed with any metabolic model, as long as the same model is used in all steps of the method according to the invention.

The methods according to the current invention can be performed with any mammalian cell, as long as a metabolic model for the cell is available or can be obtained by standard methods.

In the following an exemplary method for the generation of a metabolic model useful in the method according to the current invention is outlined.

CHO Network Model Construction, Flux Analysis, Performance Measures, and Multivariate Data Analysis

The approach as reported in Popp, O., et al. (Biotechnol. Bioeng. 113 (2016) 2005-2019; also references cited therein incorporated herein by reference) is followed as an exemplary method for generating a model to be used in the method according to the current invention. This is summarized below and outlined in the Examples section in more detail.

A genome-based CHO network model comprising five compartments (cytosol, mitochondria, ER, Golgi, bioreactor) was constructed from public sources including databases and primary literature according to established procedures and based on the approaches as outlined in the following (all incorporated herein by reference).
Oberhardt et al. (Mol. Syst. Biol. 5 (2009) 320) disclosed applications of genome-scale metabolic reconstructions. Therein it is outlined that several resources for model building and analysis exist. According to the authors, to date all high-confidence genome-scale metabolic reconstructions have been built manually through a four-step process. First, an initial reconstruction is built from gene-annotation data coupled with information from online databases such as KEGG and EXPASY, which link known genes to functional categories and help bridge the genotype-phenotype gap. Second, the initial reconstruction is curated through an examination of the primary literature. Then, the reconstruction is converted into a mathematical model that can be analyzed through constraint-based approaches. Third, the reconstruction is validated through comparison of model predictions to phenotypic data. In a final fourth step, a metabolic reconstruction is subjected to continued wet- and dry-lab cycles, which improve accuracy and allow investigation of key hypotheses.

The reconstruction includes semi-automated gene-annotation data based on BLAST-homology scores obtained from a sequenced genome, augmented by detailed, optionally manually collected data from organism-specific literature for the gap analysis during model building, whereby formerly un-annotated gene functions are incorporated into gene-annotation knowledge by analysis of incomplete but essential metabolic pathways. The gap-analysis process complemented by literature searches can reveal previously overlooked phenotypic data and pose hypotheses for enzymes that likely exist in the organism but for which no corresponding gene is currently annotated. This process serves to condense the work done on a particular organism. The gap-analysis step is also crucial for conversion of a genome-scale reconstruction as a knowledge base into the metabolic GENRE as a functional model, toward whose analysis the full suite of network tools can be applied.

It is common for reconstruction efforts to provide high-quality estimates of cellular parameters such as growth yield, specific fluxes, P/O ratio, and ATP maintenance costs, and these theoretical values are often used for hypothesis building or validation in biological studies. Excluding the two existing reconstructions of Homo sapiens metabolism the average eukaryotic network size is 800, 800, and 1300, metabolites, genes, and reactions, respectively. Between 6 and 13% of all ORFs in a eukaryotic genome are generally included in a metabolic GENRE.

Intracellular metabolic fluxes can be determined through the use of ¹³C-labeled glucose experiments, in which labeled carbon is tracked during growth of cells in a chemostat culture and computational methods are used to reconstruct the paths that carbon took inside the cells during growth. Metabolic GENREs have also been used as frameworks for interpreting metabolite concentration data. In one study, a high throughput GC-MS method was used to determine concentrations of 52 metabolites in S. cerevisiae. Differences in metabolite concentrations under known environmental conditions were mapped onto a modified S. cerevisiae metabolic GENRE, and this mapping was then combined with transcriptome data to investigate the effectors of metabolic regulation in the cell. Transcriptomic data in particular is often linked with other data types, such as protein expression data, protein-protein interaction data, protein-metabolite interaction data, and physical interaction data. Particularly in light of multiple data types, the metabolic GENRE can be a valuable tool for data interpretation.

Metabolic GENREs are best viewed as low-resolution blueprints on top of which other systems, constraints, and perturbations can be overlaid. With incorporation of regulatory and signaling data as well as other high-order systems into the constraint sets, metabolic GENREs are becoming increasingly agile and expressive of realistic cell phenotypes.

As one of the simplest and most informative methods in constraint-based modeling, FBA has become a standard in the field, with a biomass reaction usually serving as the objective. FBA predicts metabolic flux values through a network, FBA notably produces only one optimal solution, whereas it is quite common for multiple equally valid optima exist. This concept has been examined through an extension of FBA called flux variability analysis, which explores the entire optimal solution space as opposed to picking just one optimal solution, but it is an important caveat that should curb over interpretation of FBA results.

In some cases, it has been shown that knowledge of a few key parameters can be sufficient for predicting metabolic and regulatory dynamics.

Metabolic GENREs are often validated with comparisons between in silico phenotypes and various sets of in vivo data. No standard exists for how a model should be validated, which is apparent from the scattered representation of methods in validation of existing models. Recent efforts have been made to quantify the level of discrepancy expected between in silico and in vivo metabolic phenotypes. In one notable study, 465 single-gene mutants of S. cerevisiae were grown and quantified under 16 different growth conditions each. An analysis of the performance of two published S. cerevisiae metabolic GENREs revealed sensitivity (correctly predicted nonessential genes versus the total number of nonessential genes) to be on the order of 95%, and specificity (correctly predicted essentials versus the total number of essential genes) to range between 50 and 60%. These numbers were significantly improved to approximately 95-98% and 69-86% (respectively) through disqualification of some in vivo experiments, which were discovered on further analysis to be in error.

Sheikh et al. (Biotechnol. Prog. 21 (2005) 112-121) disclosed a reconstructed metabolic network. Only annotated ORFs were accounted for in the reconstructed network. Additional gene products were included on the basis of biochemical evidence in the literature. From the total gene products, unique reactions were defined. Transport processes were also accounted for further reactions. When not counting transport processes, the biggest pathways included amino acid, carbohydrate, and nucleotide metabolism. These also constituted the backbone of carbon and nitrogen metabolism. Most of the transport reactions are related to proton-linked transfer of amino acids and carbohydrates. Many of the transport reactions are inferred on the basis of physiological considerations. It is important to note that such a network is generic and does not account for differences between tissue or cell types.

Drain of metabolites for biomass synthesis was calculated based on available information in the literature on biomass composition. This information was collected from different sources investigating different cell lines, including hybridomas. An average cell composition was calculated and used for estimating requirements for each component in the biomass equation: (in %, w/w) protein, 74.2; DNA, 1.6; RNA, 6.1; carbohydrates 4.5; lipids, 10.1. An average amino acid composition was constructed. Cholesterol was the only steroid to be included in the biomass equation, as it is known to be present in significant amounts in membranes.

Growth- and non-growth-associated ATP requirements are either literature estimates or calculated from experimental data available in the literature. The efficiency of oxidative phosphorylation expressed in the P/O ratio (mol ATP produced per mol of electrons carried through the electron transport chain) was chosen to be 2.5 on the basis of literature data. Polymerization cost in terms of ATP was assumed to be the same as found for E. coli, i.e., the cost of synthesis and processing of the following macromolecules is in mol ATP/mol: protein, 4.3; RNA, 0.4; and DNA, 1.4. Multiplying the sum of amino acids, ribonucleotides, and desoxyribonucleotides with their respective cost in ATP, a total cost was calculated to be 29.2 mmol/g DW. ATP yield (YxATP) and maintenance (mATP) were estimated from a continuous hybridoma culture, assuming total ATP production can be written as a function of oxygen uptake and lactate production: r_{ATP}=r_{lac}+5*r_{O2}, where r_{ATP} is the rate of ATP production, r_{lac} is the rate of lactate production, and r_{O2} is rate of oxygen consumption. Weighted linear regression of the maintenance energy model with growth rate, μ: r_{ATP}=Yx_{ATP}*μ+m_{ATP} yielded an estimate for m_{ATP} of 1.55 mmol ATP/g DW/h and for Yx_{ATP} of 37.8 mmol ATP/g DW/h; thus, growth-associated maintenance ATP was assumed to be 8.6 mmol ATP/g DW/h.

Relatively few reactions are required for in silico growth under given constraints, which reflect the flexibility contained in the metabolic network. These are mainly involved in major catabolic pathways (glycolysis, TCA cycle, and PP pathway), nucleotide metabolism, and oxidative phosphorylation. Deleting reactions in biosynthetic pathways for biomass precursors (mainly in lipid and nucleotide metabolism) would also render the cell unable to grow. The number of essential reactions will increase once all cellular components are taken into account. Also regulation may render alternative routes infeasible in any actual cell. But, a generic in silico cell contains reactions from different cell types, which may never coexist in any given cell.

Cultured animal cells do, however, display overflow metabolism similar to that of E. coli and S. cerevisiae, suggesting a possible commonality in central carbon metabolism.

Animal cells often display a second feature known as glutaminolysis, characterized by a high glutamine uptake rate, release of ammonia by mitochondrial glutaminase, and partial oxidation of the glutamate thereby produced to alanine and/or aspartate. As the name indicates, glutaminolysis has been rationalized on an energetics basis akin to lactate production. Unlike glycolysis, however, glutaminolysis relies on the TCA cycle and oxidative phosphorylation to produce energy.

The uptake of glucose, oxygen, and glutamine was fixed at the experimentally observed rates, while lactate, ammonia, glutamate, aspartate, and alanine were left unconstrained. Glutamine synthetase was removed from the model, since murine hybridomas cannot synthesize glutamine. Similarly, the reactions for essential amino acid catabolism were removed as the uptake of essential amino acids for the hybridoma line were identical to estimated biosynthetic demands, indicating little or no catabolism. Finally, uptake or production rates for other non-essential amino acids (serine, asparagine, glycine, and proline) were fixed at actual rates, as were monoclonal antibody production rates. This was done to prevent modeling artifacts, e.g., asparagine replacing glutamine as the main nitrogen substrate.

Multiple solutions are inherently found in these simulations, specifically, for uptake of non-essential amino acids. The objective function can be achieved through several solutions of the flux distribution relating to non-essential amino acids, as these interact in many pathways (e.g., serine/glycine in glycolysis, aspartate/glutamate/glutamine/in TCA cycle, asparagins/proline in glutaminolysis).

Even though production of recombinant proteins is most likely not an objective function for the cell, it is still relevant to compare the theoretical production rate to the experimental. The maximum theoretical production rate of monoclonal antibody at different growth rates was calculated and compared to experimentally determined non-growth-associated value of 0.0084 mmol/g DW/h. In these simulations essential amino acids were unconstrained, i.e., they could be taken up freely to fulfill biomass requirements. Carbon balances were closed by constraining glucose and non-essential amino acids. Nitrogen and redox balances were closed by constraining ammonia production and oxygen uptake rate, respectively. The waste metabolism resulted in large lactate production. Glycolysis and glutaminolysis interact both in carbon and in energy metabolism. The large amount of glycolytic NADH is reoxidized by lactate dehydrogenase. NADPH is involved in glutaminolysis, where NADPH is generated in assimilation of glutamine nitrogen into biomass by glutamate dehydrogenase enzymes. Interaction of NADH and NADPH metabolism occur through transhydrogenase reaction (E.C.1.6.1.2) and isoenzymes capable of using both cofactors.

Selvarasu et al. (Biotechnol. Bioeng. 102 (2009) 923-934) used the genome-scale in silico metabolic model of E. coli iJR904. This was slightly modified to mimic the behavior of DH5a E. coli strain. The model consists of 762 metabolites (including external metabolites) and 932 biochemical reactions (including transport processes). In order to determine the metabolic fluxes, Selvarasu et al. conducted constraints-based flux analysis of the metabolic network model subjected to stoichiometric (metabolite mass balance) and thermodynamic (reaction reversibility) constraints. The residual concentration profiles of all measured nutrients and products (including glucose, trehalose, amino acids and acetate) were pre-processed to calculate their specific consumption or production rates, which were then specified as the capacity constraints in the model. The oxygen uptake rate and carbon dioxide evolution rate were unconstrained. Finally, the cellular objective of the cell growth rate during the growing phase was maximized using linear programming (LP), thereby resulting in a set of metabolic flux distribution corresponding to the optimal phenotype. Selvarasu et al. solved the LP problem by using a stand-alone flux analysis program, MetaFluxNet. The specific growth rate obtained from the optical density values (OD600) measurements during the exponential growth phase was compared with the cell growth predicted by the in silico model to validate results.

The fermentation culture was mainly explored by Selvarasu et al. during three distinct growth phases: an initial exponential growth phase characterized by high growth rate (phase 1, 1-3 h), late exponential growth phase (phase 2, 4-6 h) and acetate consumption phase (early stationary phase; phase 3, 8-10 h) in which acetate was consumed as major carbon source. In silico flux analysis was conducted for all the three phases. The specific consumption rates of all measured nutrients during phase 1 and phase 2 were ranked.

The findings of Selvarasu et al. highlight the need for accurate measurements of the highly sensitive nutrients such as arginine, serine, glucose and trehalose in the complex medium since they play an important role in the functioning of cellular metabolism. Such measurements may also provide crucial information for designing efficient media components.

Based on the flux distribution, the summation of all the incoming or outgoing fluxes (flux-sum) around a particular metabolite was calculated in order to analyze its consumption and production within the cell.

Although the in silico metabolic model reported by Selvarasu et al. predicted the cell growth rate reasonably well, prediction can be further improved by considering other important metabolites in the supplement medium. This also indicates the need for defining and accurately measuring other key metabolites in order to precisely evaluate cellular metabolism under complex medium condition.

The phenotypic state and metabolic behavior during early stationary phase can be best characterized by minimizing ATP flux, while constraining the growth rate and consumption/production rates of other nutrients/products to the experimental values. Nevertheless, the resultant simulated metabolic fluxes must be qualitatively or quantitatively validated by comparing the simulated metabolic behavior with internal flux changes derived from gene expression profiles or with experimentally determined fluxes.

Selvarasu et al. (Mol. Biosyst. 6 (2010) 152-161) reported about a genome-scale reconstruction of mouse metabolic network. The genome-scale metabolic network of mouse was systematically reconstructed based on a previous model and relevant information from various resources.

A previous generic model of mouse26 was considered by Selvarasu et al. as a starting point. Initially the repeated or redundant reactions in the model were identified and removed. Then, various simulations of the model were performed to verify its ability to produce each cellular component defining the biomass from different carbon sources. This allowed Selvarasu et al. to find missing links or gaps in the network and subsequently fill them by adding relevant enzymatic and transport reactions obtained from several online resources (KEGG, RIKEN, MGI, BRENDA, and ExPaSy) and relevant literature to M. musculus. Additionally, information on new open reading frames (ORFs) and GPR association were also included, thus significantly expanding the scope of the model.

The visualization and statistical analysis of reconstructed genome-scale mouse network in Selvarasu et al. were all performed using the network analysis software, BioNetMiner (http://bio.netminer.com). A large-size mouse network can be efficiently visualized by BioNetMiner embedding graph layout algorithms, Force-Directed Kamada-Kawai and GEM. In addition, the network topology can be statistically analyzed by identifying highly-connected and bridging metabolites using degree and betweenness centrality, respectively.

Once reconstructed genome-scale metabolic network is stoichiometrically balanced, the predictive capabilities of the model can be examined in both quantitative and qualitative manners by resorting to constraints-based flux analysis. Initially, under stationary assumption during cell growing phase, cell biomass production can be considered as plausible cellular objective to be maximized for quantifying the cellular growth phenotype. The resulting growth rate is then compared with experimentally observed specific growth rate. Subsequently, the model can be qualitatively assessed by simulating minimal media requirements and gene deletion analysis. The minimal nutrient components can be determined by minimizing the summation of all consumed substrates from the medium; under the determined minimal medium condition, the cell growth was maximized, constraining each reaction flux to be zero. The reaction and corresponding gene were deemed essential when their removal resulted in zero growth. Similarly, essential metabolites can be identified by forcing the flux sum across each metabolite as zero under cell growth condition. Finally, the functional organization of the mouse metabolism can be investigated on the basis of gene/metabolite essentiality and its correlation with structural characteristics of the network. All these linear optimization problems were solved by MetaFluxNet and GAMS/CPLEX 10.0.

Compared to the previous model of mouse26, Selvarasu et al. newly added 490 reactions, providing updated information on gene-protein-reaction (GPR) association and detailed description on lipid, amino acids, carbohydrate and nucleotide metabolisms. The model is comprised of 724 genes, 715 enzymes, 1162 internal metabolites, and 1494 reactions; 1246 reactions are biochemical conversions within cytosol (1085) and mitochondria (161), and 248 are exchange reactions describing the metabolite transport between intra- and extra-cellular membrane (171) and cytosol and mitochondria (77). In addition to biochemical reactions, Selvarasu et al. derived one balance equation for expressing the cell biomass from the drain of biosynthetic precursors such as proteins, lipids, carbohydrates, DNA, RNA, and other cellular components at their experimental composition and relevant energy cofactors for their conversion and assembly. During the reconstruction process, manual curation of the resulting network was iteratively performed by checking the consistency, accuracy, and completeness of the model until simulated results were consistent with experimental observation both quantitatively and qualitatively. It allowed Selvarasu et al. to find knowledge gaps for refining the model.

The predictive capability of the mouse model was tested using constraints-based flux analysis, based on batch cultural data of mouse hybridoma cells producing anti-F monoclonal antibody, grown in a DMEM media supplemented with proline, asparagine and aspartate. The biomass production was maximized to simulate the cell growth condition, constraining the measured specific consumption/production rates of nutrients/products during the culture. The resultant growth rate (0.048 h⁻¹) was higher than the average specific growth rate (0.0362 h⁻¹) in the entire batch culture. Selvarasu et al. believed that the growth prediction can be improved when relevant measurements for in silico simulation are used to reflect more realistic operational condition during exponential growth phase.

For qualitative model prediction, Selvarasu et al. conducted in silico analysis on minimal media requirements for cell growth and finally identified required medium components. Selvarasu et al. include essential amino acids, folate and phosphate which are almost consistent with experimentally observed essential components and the nutrition requirements for laboratory animals. However, in silico analysis could not identify some minimal medium components such as growth factors, cofactors, and minerals (biotin, thiamine, vitamins, calcium and magnesium ions, etc.). Not surprisingly, the predicted growth of the mouse cell was not directly affected only by glucose uptake. Instead, it was determined by the uptake of essential amino acids, thus confirming previous observation that under glucose-deprived or limited conditions, unlike microbial cells mammalian system can survive by utilizing other nutrients like essential amino acids.

Gene essentiality analysis also allowed Selvarasu et al. to validate and improve the new mouse model in an iterative way. All predicted essential genes using current and previous models were compared with experimentally reported essential genes from KOMP (KnockOut Mouse Project) database. Most in silico essential genes are experimentally confirmed while Selvarasu et al. also found some false positive predictions. Such information can be newly included in the model to improve its predictions.

The characteristic features of the reconstructed model were explored from its structural and functional points of view. First, the statistical network analysis identified a large cluster of weakly connected reactions (89% of total reactions) and 119 small clusters with 1 to 17 connecting reactions. Selvarasu et al. then calculated the network diameter while the cofactor metabolites (e.g., ATP, H₂O, CO₂, etc.) were excluded to prevent biologically meaningless results of identifying them as major hubs in the network. The resulting network diameter for the large cluster was measured to be 40. The average path length (APL) was also calculated as 8.51, revealing that most of the metabolites in the network can be converted between each other by approximately 3B4 reactions. Similar analysis was conducted for three major sub-networks, which were significantly improved from the previous model, carbohydrate, amino acids and lipid metabolisms, resulting in different network diameters and APLs.

Selvarasu et al. also explored the network topology by calculating degree and betweenness centrality of metabolites, thus identifying highly connected and critical (bridge-acting) components within the network. Selvarasu et al. further investigated the topological properties of the network by comparing the essential metabolites with their centrality scores. The essential metabolites for the cell growth were obtained using flux sum approach. It was observed that the average centrality scores of essential metabolites (degree: 6.37 and betweenness centrality: 0.00198) were much higher than the non-essential ones (degree 2.55 and betweenness centrality: 0.00039). Unexpectedly, metabolite centrality was not clearly correlated with metabolite essentiality.

Selvarasu et al. identified a set of essential genes for the cell growth in a defined medium. Initially, single-gene reaction association was assumed to perform gene deletion analysis under rich medium (RM) as well as minimal medium (MM) conditions. Of 109 essential reactions under RM condition, 93 were gene-associated, 6 non-gene-associated, and 10 for the transport of amino acids. Interestingly, the highest percentage (59%) of essential reactions is from lipid metabolism (fatty acid biosynthesis and fatty acid metabolism), indicating that it may be one of the most vulnerable sub-systems to environmental disturbances. The additional 6 reactions under MM condition are from amino acids (5) and carbohydrate (1) metabolism. When GPR associations were considered, only 72 essential genes were identified as there were many isozymes and multifunctional proteins in the current genome-scale model. For example, fatty acids synthase (fasN), one of the multifunctional proteins, alone catalyzed 37 reactions in lipid metabolism, while other genes or proteins are associated with at least two or more reactions in the metabolic network.

The presence of low percentage (10%) of essential reactions implies that mouse metabolism is highly flexible and robust upon internal changes to attain the same phenotype through alternate pathways, thus rendering two reactions/genes non-essential and making the network flexible. In the view of exploring such combinatorial genes/reactions, Selvarasu et al. conducted double-knockout analysis. From more than 9.5*10⁶ pairs of 1385 non-essential reactions, Selvarasu et al. could identify only 139 lethal pairs involving 114 unique reactions. Most essential pairs belong to two categories: (i) two reactions producing the same metabolite, and (ii) subsequent two reactions producing and consuming same metabolite. Similar analysis has been successfully applied and the functional features have been elucidated for H. pylori, as such demonstrating the cellular robustness and suggesting multiple deletion analysis for identifying drug targets.

In the exemplary model (see Materials and Methods in the Examples section below) used for demonstrating the current invention central metabolic pathways (glycolysis, citric acid cycle, pentose phosphate pathway, respiratory chain) and in addition also the biosynthesis of major biomass constituents (protein, lipid, RNA, DNA, carbohydrates), C₁-metabolism, and amino acid degradation pathways have been included. For recombinant protein product formation investigation, a corresponding set of product formation reactions was formulated, which accounted for the amino acid composition and a representative glycosylation structure (two sialilated biantennary glycans per product molecule) of the recombinant protein. The resulting model comprised 654 reactions, 583 metabolites, and represented 266 ORFs. The biomass composition was chosen comparable to previous studies for CHO cells or murine cell lines (see e.g. Altamirano et al., Biotechnol. Prog. 17 (2001) 1032-1041; Bonarius et al., Biotechnol. Bioeng. 50 (1996) 299-318; Selvarasu et al., Biotechnol. Bioeng. 109 (2012) 1415-1429).

Model reconstruction and model simulations were performed using a commercially available software package. For model verification, it was confirmed that the elemental balance and charge balance is closed for all reactions. Moreover, Flux Balance Analysis (see e.g. Savinell and Paulson, J. Theor. Biol. 154 (1992) 421-454 and 455-473) was used to verify functionality of individual pathways. Time-series transcript data collected during CHO fermentations served to delineate (in)active metabolic routes in the network and supported identification of predominant isoenzyme species.

Estimation of cellular uptake and production rates was performed by first subdividing the whole fermentation process into physiologically distinct process phases. This can be done, for example, through a computational optimization procedure, wherein the optimum number of process phases is determined using a χ²-based goodness-of-fit test. During each process phase, constant cell physiology was assumed, implying constant biomass-specific rates. These biomass-specific rates were determined using non-linear regression. The resulting uptake and production rates may serve as inputs for performing metabolic flux analysis (see e.g. Maier et al., Biotechnol. Bioeng. 100 (2008) 355-370; Niklas et al., Curr. Opin. Biotechnol. 21 (2010) 63-69; Stephanopoulos et al., 1998, Metabolic engineering: Principles and methodologies. San Diego: Academic Press). Thermodynamic consistency of the computed flux distributions was confirmed.

Computation of Pearson and Spearman correlations of metabolite data, process data, and of intracellular flux distributions were performed using a commercially available software package (see Materials and Methods in the Examples section below).

### EMBODIMENTS OF THE INVENTION

Mechanistic metabolic modeling can be useful for:
efficient and fast selection/evaluation of high producer clones,
increase of volumetric titer by modulation/optimization of media composition, feeding regime, and process parameters,
increase of product quality by modulation/optimization of media composition, feeding regime, and process parameters, and/or
integration of high-throughput data and condensation into a readable and interpretable format.

Mechanistic modelling allows for a temporal resolution and analysis of intracellular metabolic fluxes (MFA) and their optimization (FBA). The overall goal of mechanistic modelling is a high-throughput method for automated CHO cell performance analysis and thereby allowing for process optimization and/or clone selection.

But the method needs reliable and consistent input (raw) data to provide useful and reliable results/read-out.

It has now been found by the current inventors that mechanistic metabolic modelling can also be used for quality control of high-throughput cultivation data, such as
identification of technical problems during a cultivation run, such as e.g. probe shut down, sensor drift, plugged pipes, in-process-control analytic errors, offline analytical errors, culture media preparation issues, etc.,
and/or
pre-processing/data consistency check in process control.

Thus, the current invention comprises methods for efficient, consistent, and optionally user-independent data consistency check for any in-process and final cultivation data. The methods according to the invention are especially suitable for high-throughput application.

Herein is reported the use of generic models, i.e. one that can e.g. be used for all CHO clones and CHO-based processes as well as E. coli clones and E. coli-based processes, as point of efficient data integration for fermentation data from various different in-line, at-line and off-line data as well as interpretation and/or data analysis. The model allows for cross-checking of data and reduces the degree of freedom in the data (“Does the value makes sense?”). A lack-of-fit in the data is used to identify corrupted/inconsistent data source(s) (e.g. defect sensor, missing data, human errors, etc.). Thereby it becomes possible to use mechanistic metabolic modeling for more reliable, more efficient and fast identification and/or selection and/or evaluation of high producer clones; screening processes for increasing volumetric titer by modulation/optimization of media composition, feeding regime, and process parameters; identification of technical problems during cultivations (e.g. probe shut down), IPK analytics; integration of high-throughput data and condensation into readable and interpretable format; reliable knowledge generation; or/and pre-processing/data consistency check in process control.

In one example, different CHO—K1 cell clones, all stably expressing the same recombinant monoclonal IgG4 antibody, were used. Cultures were sampled daily to perform comprehensive metabolic profiling.

In order to assess metabolic cell performance throughout the cultivations in detail a CHO metabolic network model was employed. Each cultivation was subdivided into five physiologically and metabolically distinct process phases, phase 1 (Ph1) to phase 5 (Ph5) as listed in the following Table 1. Cellular uptake and production rates were determined as described in Example 1.

TABLE 1
Metabolic phases of host cell line (HCL) and recombinant
CHO clones employed in the exemplary metabolic model.
Distinct metabolic phases of HCL and recombinant CHO
clones were identified (HCL and 10 clones, 3 data sets).

| | phase 1 | phase 2 | phase 3 | phase 4 | phase 5 |
|---|---|---|---|---|---|
| cell line/clone | [days] | [days] | [days] | [days] | [days] |
| parental cell line | 0-3 | 3-7 | 7-9 | 9-11 | 11-13 |
| clone 4 | 0-5 | 5-7 | 7-9 | 9-11 | 11-13 |
| clone 5 | 0-3 | 3-6 | 6-9 | 9-11 | 11-13 |
| clone 6 | 0-5 | 5-7 | 7-9 | 9-11 | 11-13 |
| clone 7 | 0-4 | 4-6.5 | 6.5-9 | 9-11 | 11-13 |
| clone 8 | 0-5 | 5-7 | 7-9 | 9-11 | 11-13 |
| clone 9 | 0-3 | 3-6 | 6-8 | 8-12 | 12-13 |
| clone 10 | 0-3 | 3-6 | 6-9 | 9-12 | 12-13 |
| clone 11 | 0-5 | 5-7 | 7-10 | 10-12 | 12-13 |
| clone 12 | 0-3 | 3-6 | 6-8 | 8-9.5 | 9.5-13 |
| clone 13 | 0-2.5 | 2.5-6 | 6-10 | 10-12 | 12-13 |

This step included mass balancing of the whole process including feeding and sampling events. For each process phase, intracellular flux distributions were calculated using the network model. By accounting for time-dependent changes in cell physiology and not relying on end-point data alone, this resulted in a comprehensive characterization of each cell/clone/phase.

By using normalized scores, indicator types (such as extracellular concentration measurements, uptake rates, and intracellular fluxes) can be integrated into a combined scoring scheme. In addition, based on the availability of time-series data, the quantification of individual indicators on that part of the process where they are most relevant was possible by assigning time-dependent scores. For example, fast cell growth was scored as more important early in the process while high specific productivity was of special interest after day 6, i.e. once high cell numbers had been established and where the majority of product formation occurred.

In total, about 40 different indicators were defined to characterize the metabolic performance of the CHO cells and clones regarding recombinant protein and biomass formation and metabolic efficiency (see the following Table 2).

TABLE 2
Metabolic performance indicators defined. Six metabolic performance criteria were defined as major
hallmarks of CHO metabolism by clustering selected metabolic performance parameters calculated
by the CHO network model: “Product Formation”, “Cell Growth”, “Lactate Formation”, “Ammonium
Formation”, “Metabolic Clone Efficiency”, and “Respiration”. The rank order describes if a high
(“1”) or low (“0”) level of the performance indicator is favored.

| metabolic | metabolic | unit (before | | indicator | | | | | |
|---|---|---|---|---|---|---|---|---|---|
| performance | performance | normali- | rank | weight | | | | | |
| criterion | indicator | zation) | order | W_{i,j}^{IND} | Wₚₕ₁^{IND i,j} | Wₚₕ₂^{IND i,j} | Wₚₕ₃^{IND i,j} | Wₚₕ₄^{IND i,j} | Wₚₕ₅^{IND i,j} |
| product formation | | | | | | | | | |
| rationally describes | max. product | mg/L | 1 | 0.333 | 0.100 | 0.100 | 0.200 | 0.300 | 0.300 |
| the clone product | titer in | | | | | | | | |
| formation capacity and | metabolic phase | | | | | | | | |
| metabolic-economic | specific | pg/(cell · d) | 1 | 0.333 | 0.125 | 0.125 | 0.250 | 0.250 | 0.250 |
| efficiency (substrate | productivity | | | | | | | | |
| utilization for | product titer | mg/L | 1 | 0.333 | 0.111 | 0.222 | 0.222 | 0.222 | 0.222 |
| product formation) | increase in | | | | | | | | |
| | metabolic phase | | | | | | | | |
| cell growth | | | | | | | | | |
| rationally describes | apparent | 1/d | 1 | 0.214 | 0.286 | 0.286 | 0.143 | 0.143 | 0.143 |
| the biomass formation | specific | | | | | | | | |
| capacity “active” | growth rate μ | | | | | | | | |
| biomass content and | (DW-based) | | | | | | | | |
| metabolic-economic | max viable | 10⁵ | 1 | 0.214 | 0.111 | 0.222 | 0.222 | 0.222 | 0.222 |
| efficiency (substrate | cell conc. in | cells/mL | | | | | | | |
| utilization for | metabolic phase | | | | | | | | |
| biomass formation) | vViable cell | 10⁹ cells | 1 | 0.143 | 0.286 | 0.286 | 0.143 | 0.143 | 0.143 |
| | production in | | | | | | | | |
| | metabolic phase | | | | | | | | |
| | IVCD | 10⁹ cells/L · d | 1 | 0.071 | 0.200 | 0.200 | 0.200 | 0.200 | 0.200 |
| | minimum | % | 1 | 0.143 | 0.111 | 0.111 | 0.111 | 0.333 | 0.333 |
| | viability in | | | | | | | | |
| | metabolic phase | | | | | | | | |
| | estimated | 1/d | 0 | 0.214 | 0.286 | 0.286 | 0.143 | 0.143 | 0.143 |
| | specific death | | | | | | | | |
| | rate | | | | | | | | |
| lactate formation | | | | | | | | | |
| rationally describes | lactate | μmol Lactate/ | 0 | 0.250 | 0.250 | 0.250 | 0.250 | 0.125 | 0.125 |
| the lactate formaton | production | (10⁹ cells · h) | | | | | | | |
| cpacity and kinetics | max. lactate | mM | 0 | 0.500 | 0.250 | 0.250 | 0.250 | 0.125 | 0.125 |
| | conc. in | | | | | | | | |
| | metabolic | | | | | | | | |
| | phase | | | | | | | | |
| | lactate conc. | mM | 0 | 0.250 | 0.250 | 0.250 | 0.250 | 0.125 | 0.125 |
| | increase in | | | | | | | | |
| | metabolic | | | | | | | | |
| | phase | | | | | | | | |
| ammonium formation | | | | | | | | | |
| rationally describes | NH₄ | μmol NH₄/ | 0 | 0.200 | 0.250 | 0.250 | 0.250 | 0.125 | 0.125 |
| the ammonium | excretion | (10⁹ cells · h) | | | | | | | |
| formation capacity | substrate | % Nmol | 1 | 0.200 | 0.200 | 0.200 | 0.200 | 0.200 | 0.200 |
| and kinetics | fraction NH₄ | substate | | | | | | | |
| | max. NH₄ | mM | 0 | 0.200 | 0.250 | 0.250 | 0.250 | 0.125 | 0.025 |
| | concentration | | | | | | | | |
| | in metabolic | | | | | | | | |
| | phase | | | | | | | | |
| | NH₄ conc. | mM | 0 | 0.400 | 0.250 | 0.250 | 0.250 | 0.125 | 0.125 |
| | increase in | | | | | | | | |
| | metabolic phase | | | | | | | | |
| metabolic clone efficiency | | | | | | | | | |
| rationally describes the | biomass yield | Cmol biomass/ | 1 | 0.063 | 0.286 | 0.286 | 0.143 | 0.143 | 0.143 |
| metabolic efficiency of | Cmol | Cmol | | | | | | | |
| a clone | | substrates | | | | | | | |
| | biomass yield | Nmol biomass/ | 1 | 0.063 | 0.286 | 0.286 | 0.143 | 0.143 | 0.143 |
| | Nmol | Nmol | | | | | | | |
| | | substrates | | | | | | | |
| | product yield | Cmol | 1 | 0.063 | 0.125 | 0.125 | 0.250 | 0.250 | 0.250 |
| | Cmol | product/ | | | | | | | |
| | | Cmol | | | | | | | |
| | | substrates | | | | | | | |
| | product yield | Nmol | 1 | 0.063 | 0.125 | 0.125 | 0.250 | 0.250 | 0.250 |
| | Nmol | product/ | | | | | | | |
| | | Nmol | | | | | | | |
| | | substrates | | | | | | | |
| | est.ATP for | μmol/(10⁹ | 0 | 0.125 | 0.125 | 0.125 | 0.250 | 0.250 | 0.250 |
| | maintenance | cells · h) | | | | | | | |
| | total Cmol | μmol/(gDW · h) | 1 | 0.125 | 0.200 | 0.200 | 0.200 | 0.200 | 0.200 |
| | flux | | | | | | | | |
| | total Nmol | μmol/(gDW · h) | 0 | 0.125 | 0.200 | 0.200 | 0.200 | 0.200 | 0.200 |
| | flux | | | | | | | | |
| | fraction of ATP | % of total | 1 | 0.125 | 0.250 | 0.250 | 0.250 | 0.125 | 0.125 |
| | for cell protein | ATP synthesized | | | | | | | |
| | translation | | | | | | | | |
| | fraction of ATP | % of total | 1 | 0.125 | 0.125 | 0.125 | 0.250 | 0.250 | 0.250 |
| | for product | ATP synthesized | | | | | | | |
| | translation | | | | | | | | |
| | total ATP | μmol/(10⁹ | 1 | 0.125 | 0.200 | 0.200 | 0.200 | 0.200 | 0.200 |
| | production | cells · h) | | | | | | | |
| respiration | | | | | | | | | |
| rationally describes the | specific O₂ | μmol/(10⁹ | 0 | 0.333 | 0.200 | 0.200 | 0.200 | 0.200 | 0.200 |
| respiration of a clone as | uptake | cells · h) | | | | | | | |
| a measure of metabolic- | specific CO₂ | μmol/(10⁹ | 0 | 0.333 | 0.200 | 0.200 | 0.200 | 0.200 | 0.200 |
| economic usage of | production | cells · h) | | | | | | | |
| substrates and formation | RQ | (—) | 0 | 0.333 | 0.200 | 0.200 | 0.200 | 0.200 | 0.200 |
| of by products | | | | | | | | | |

Since certain traits of interest were characterized by more than one indicator (e.g., both, a high end titer and high specific productivity would be desirable for a good producer clone) related indicators are grouped into distinct categories: product formation, cell growth, lactate formation, ammonium release, respiratory metabolism, and metabolic clone efficiency.

A good model for clone characterization has to meet several requirements: (i) sensitive discrimination between clones for performance criteria, (ii) comprehensive characterization of clone traits, and (iii) robustness of the assessment procedure to level normal variations in cultivation runs.

Regarding the first aim, product titer and cell growth routinely serve as sensitive measures of clone performance. Using the coefficient of variation as variance measure, the category “metabolic clone efficiency” also contributed sensitive clone characterization criteria whereas lactate formation and respiratory metabolism were less informative. Nevertheless, they contribute to comprehensive clone characterization as they reflect cellular properties that have a major impact on process performance especially at larger scales.

Extracellular metabolite data as well as intracellular flux distributions served to compute pre-defined measures of metabolic cell performance including product titer, the integral of viable cell density (IVCD), and specific productivity, but also carbon yields of biomass and product formation, rates of intracellular glutamine synthetase, and predicted ATP requirement for maintenance.

Thus, the model is based on the experience of utilizing in-depth metabolic analysis of CHO cultures. It is an integral multi-level workflow for the mechanistic characterization and identification of recombinant CHO clones and process variations. More specifically, the model is applicable to small-scale cultivations in shaker flasks or multi-well plates. Likewise, controlled multiplex small-scale bioreactors and in-depth high-throughput analytics for determining process parameters, key metabolic performance markers, and critical product quality attributes can be used.

In the current invention a metabolic network simulation environment is applied to CHO clones' characterization and high-throughput data validation.

It has been found that the same mechanistic model used to simulate intracellular biochemical reactions based on extracellular measurements (such as, e.g., 2-Oxoglutarate, 5-Oxoproline, Acetate, beta-Alanine, beta-D-Glucose, beta-Methylnorleucine, Biomass, Butyrate, Choline, Citrate, CO₂, D-Gluconate, D-Mannose, Ethanolamine, Formate, Fumarate, GABA, Glycine, H2S, potassium, L-Alanine, L-Arginine, L-Asparagine, L-Aspartate, L-Citrulline, L-Cysteine, L-Cystine, L-Glutamate, L-Glutamine, L-Histidine, L-Isoleucine, L-Lactate, L-Leucine, L-Lysine, L-Methionine, L-Ornithine, L-Phenylalanine, L-Proline, L-Serine, L-Threonine, L-Tryptophan, L-Tyrosine, L-Valine, myo-Inositol, N-Acetylputrescine, N1-Acetylspermidine, N1-Acetylspermine, N8-Acetylspermidine, sodium, O₂, phosphate, Orthophosphate, Product, Putrescine, Pyruvate, Spermidine, Spermine, Succinate, Sulfate, Uridine, etc.) once established and proven to be appropriate for mirroring cellular processes in silico can be used for the validation or/and consistency check of in-process recorded data of new cultivation data sets.

High-throughput screening (HTS) in-process data fitted using an existing metabolic model showed erratically large deviation of model fit quality, i.e. based on the model calculated fitted lines were deviating dramatically from the experimental data, i.e. were badly fitted. There can be different reasons for such a bad fit, such as, e.g., clone variances, data (in)consistency, technical problems, etc.

Of these reasons technical problems are the most dangerous as thereby potentially suitable clones are discarded. Amongst other things technical problems could be, e.g., no off-gas (CO₂, O₂), pH-sensor drift during cultivation, plugged pipes and no feed added despite pump working, no debris measured, unmeasured metabolites (e.g. organic acids and precursors thereof, polyamines and precursors thereof, sugars and precursors thereof, activated sugars and precursors thereof, nucleotides and precursors thereof, nucleosides and precursors thereof, redox equivalents and precursors thereof, redox active compounds and precursors thereof, lipids and precursors thereof, endogenous host proteins, etc.) no sample drawing, inaccurate measurements of biomass or metabolites, analytical errors resulting in wrong values, etc.

In FIG. 1A and FIG. 1B the effect of an exemplary technical problem resulting from incomplete feed data is shown. In FIG. 1A the analysis of a cultivation based on incomplete feed data is shown. In FIG. 1B the analysis of the same cultivation with completed feed data is shown. It can clearly be seen that due to the incomplete data the resulting fit based on the metabolic model is bad (bad model fit is indicated by offset of modeled fits (line) vs. raw data (boxes)). Without questioning or checking the data this would suggest that the respective clone does not behave well. But actually the data entry was erroneous, i.e. a wrong glucose concentration had been entered. If no check of the data for data consistency is carried out this issue will not be discovered.

A typically fermentation data set spans a period of two weeks with daily data points for about 15 parameters on-line and about 30 parameters off-line. This process data set is influenced by the biological variance of the cell clone as well as by the process variance of the employed devices and the cultivation method.

Biological variance stems from clone-to-clone difference in, e.g., biomass accumulation, product formation (rates), nutrient consumption (rates), waste product formation (rates), or cell viability robustness.

Process variance reflects the technical fluctuations within the tolerance range, e.g., of the start concentrations, in vessel size/geometry, in temperature, in stirring speed and uniformity, in gassing, in feeding, in mass/volume balancing, in addition/amount of correction agents.

The combination of biological and technical variance is reflected in the process data.

For the generation of a reliable metabolic model input data obtained with a broad spectrum of clones, products, scales, data density, host cell lines, and cultivation platforms is used.

An exemplary data set used for the assessment of data quality is shown in the following Table 3.

TABLE 3
Overview table of 51 different cultivation experiments (batches)
for model development. “HCP”: host cell protein; “+”: data available;
“−”: data not available; “na”: not applicable since host cell line(s),
producing no product

| batch | | | | | off |
|---|---|---|---|---|---|
| (n = 1-3) | product | scale | | HCP | gas |
| 1/2 | B | 2 | L | +/+ | −/− |
| 3/4/5 | B | 2 | L | +/+/+ | −/−/− |
| 6/7 | B | 2 | L | +/+ | −/− |
| 8/9 | B | 2 | L | +/+ | −/− |
| 10/11 | B | 2 | L | +/+ | −/− |
| 12 | D | 2 | L | + | − |
| 13/14 | D | 250 | mL | +/+ | +/+ |
| 15 | D | 2 | L | + | − |
| 16/17 | D | 250 | mL | +/+ | +/+ |
| 18 | D | 2 | L | + | − |
| 19/20 | D | 250 | mL | +/+ | +/+ |
| 21/22/23 | A | 250 | L | +/+/− | −/−/− |
| 24/25 | E | 2 | L | +/+ | −/− |
| 26 | E | 2 | L | + | − |
| 27/28 | A | 2 | L | +/+ | −/− |
| 29/30 | na | 2 | L | +/+ | −/− |
| 31/32/33 | na | 2 | L | +/+/+ | −/−/− |
| 34 | F | 2 | L | − | − |
| 35 | F | 2 | L | − | − |
| 36 | F | 2 | L | − | − |
| 37 | F | 2 | L | − | − |
| 38 | G | 2 | L | − | − |
| 39 | H | 2 | L | − | − |
| 40/41 | D | 15 | mL | −/− | −/− |
| 42/43 | D | 15 | mL | −/− | −/− |
| 44/45 | D | 15 | mL | −/− | −/− |
| 46/47 | D | 250 | L | −/− | −/− |
| 48/49 | D | 1000 | L | −/− | −/− |
| 50/51 | D | 2000 | L | −/− | −/− |

With the replicates as shown in the table above the model was trained and adopted. Thereby it was possible to identify runs that had an inconsistency. The analysis is shown exemplarily for runs 9, 14 and 51 in FIG. 2.

It can be seen that fermentation 51 is inconsistent with the model as some data points are deviating from the 1:1 line. This offset from the 1:1 line indicate bad data consistency for the respective parameter.

This analysis has been re-done for multiple fermentations. The respective data and its correlation in shown in FIG. 3A and FIG. 3B. Fermentations with high χ² (chi\hat{ }2) values>5 or close to 0 (e.g. <0.1) failed in the respective used model variant. It can been seen that certain runs show inconsistencies that are not based on the cultivated clone, but on experimental, technical defects.

Without being limited by this explanation, some inconsistencies are due to a limited number of data points (less than 3 per cultivation phase or less than 6 overall), due to technical problems with the on-line or at-line analytical devices, or due to errors in HCP (host cell protein) or OUR (oxygen uptake rate) determination by off-gas analytics.

The method according to the current invention can be used to identify inconsistent, i.e. wrong, input data. This is shown in the following example, wherein erroneous off-gas measurements resulted in a deviation between experiment and model prediction.

The chi²-value is used for determining the quality of the fit between model and experiment for the respective parameter. This analysis revealed that for all scenarios except one the chi²-value was in the same range. In the exceptional scenario oxygen uptake measurements were included in the analysis (see Table 4). The lack of fit of those data could be resolved by identifying the underlying reason. After resolving this inconsistency, the chi²-value was acceptable for all studied scenarios.

TABLE 4
Median chi²-values analyzed for each scenario. For scenario
“model 1” a dataset with wrong and curated OUR data were used.
For all other scenarios the curated OUR data set were used.

| Scenario | OUR | chi² |
|---|---|---|
| Model 1 | erroneous data | 94.8 |
| Model 1 | corrected data | 1.3 |
| Model 2 | corrected data | 0.7 |
| Model 3 | corrected data | 0.8 |
| Model 4 | corrected data | 0.5 |
| Model 5 | corrected data | 0.5 |

From the chi²-value of scenario “model 1” with wrong OUR data it can be seen that there is a bad fit between model and experiment. An acceptable model fit is obtained when the corrected data is used.

Oxygen is a key substrate in animal cell metabolism. It has been reported that the oxygen uptake rate (OUR) is a good indicator of cellular activity, and even under some conditions, a good indicator of the number of viable cells. The measurement of OUR is difficult due to many different reasons. In particular, the very low specific consumption rate (0.2×10⁻¹² mol cell h⁻¹), the sensitivity of the cells to variations in dissolved oxygen concentration and the difficulty to provide oxygen without damaging the cells are problems which must be taken into account for the development of OUR measurement methods. Different solutions based on an oxygen balance on either the liquid phase or around the entire reactor, and with a variable or stable concentration of dissolved oxygen have been reported. To determine OUR, one of the two following approaches is generally used. It is possible to consider the whole reactor (equation (1)) or only the liquid (equation (2)) phase to write the oxygen mass balance:

$$\begin{matrix}\frac{{dC}_{L}}{dt} \cdot {V}_{L} + \frac{d ( {C}_{G} \cdot {V}_{G} )}{dt} = {G}_{in} \cdot {y}_{in} - {G}_{out} \cdot {y}_{out} - OUR \cdot {V}_{L} & ( 1 ) \\ \frac{{dC}_{L}}{dt} = {k}_{L} a ( \frac{{P}_{tot} \cdot {y}_{G}}{H} - {C}_{L} ) - OUR & ( 2 )\end{matrix}$$

(see Ruffieux, P-A., et al., J. Biotechnol. 63 (1998) 85-95).

Thus, the OUR is no directly measurable value. It is dependent on different additional variables and requires calculation.

By reviewing the input data in the current case it turned out that the mathematical equation used for the calculation contained an error (wrong factor of 1000). After identification thereof based on the method according to the invention the data was reprocessed with the curated equation resulting in an improved chi²-value as shown in the Table 4 (see above).

The method according to the current invention can on the one hand identify technical and operational problems during data generation as outlined above and also verify the correctness of input and calculated data. Thereby confirmation is provided that determined data is actually a property of the respective clone, i.e. its phenotype, and not due to a technical or operational error. This is shown in the following example.

Two clones were cultivation under the same cultivation conditions. Whereas the viable cell density was comparable (see FIG. 4A) between clone 1 and clone 2 the lactate concentration in the cultivation medium was different (see FIG. 4B). It can now be questioned if the low lactate levels observed for clone 2 are reliable and, thus, a property of the clone's geno/phenotype. By using the method according to the invention it can be established that the phenotype of the second clone is correctly reflected by the data and not due to technical deviations or problems. This is exemplified by the analysis using the corresponding metabolic model. The good model fits (agreement of reconciled and black box rates, see FIG. 5) for all metabolic phases 1 to 5 show feasibility of measured rates. The low level of lactate can be described meaningfully by the mechanistic metabolic modeling.

Thus, the current invention comprises methods for
selecting/evaluating cell clones,
increase volumetric titer of cell clones by modulation/optimization of media composition, feeding regime, and process parameters,
increase of product quality by modulation/optimization of media composition, feeding regime, and process parameters,
identifying of technical problems during a cultivation run or in-process-control analytics,
checking pre-processing/data consistency in process control.

The essential element of these methods is the same: the control of data sets using the fit to a mechanistic model of the respective cell line.

It is an objective of the present invention to provide an improved method for determining if cultivation process data are affected by a technical problem as specified in the independent claims and aspects as outlined herein. Embodiments of the invention are given in the dependent claims. Embodiments of the present invention can be freely combined with each other if they are not mutually exclusive.

During the cultivation of cells, e.g. for producing byproducts, time-dependent, i.e. temporal, process data is acquired and archived. This process data is the sum of on-line and off-line temporal process parameters. Thus, the process data reflects the time dynamics of the respective process parameters and outcome variables, such as e.g. specific rates. The outcome variables are often obtained in a pre-processing step involving, e.g., transformation, normalization, integration, and computation of missing values.

The cultivation devices are typically equipped with automated control and data logging systems whereby acquired process data are recorded and archived on-line electronically. The acquired on-line process parameters include control parameters and control action parameters. The control parameters include parameters such as dissolved oxygen (DO), pH, and vessel temperature that are controlled at specific levels (e.g., vessel temperature at 37° C.), whereas the control action parameters include parameters such as controller responses, the sparge rates of air and oxygen to control DO, and the rates of base addition and carbon dioxide sparge to control pH. Other important parameters such as vessel volume and overlay gas flow rates are also acquired on-line. The volumetric oxygen uptake rate (OUR) is estimated approximately every 4 hours, whereas all other on-line parameters are acquired almost continuously (at least daily and down to once every few seconds) over the entire duration of the run that lasts several days. In addition to these parameters whose values are continuous, there are ‘discrete’ parameters such as the state of different valves, which is often binary (“OFF/ON” state). These valves control different ports for addition of inoculum, media, base, anti-foam, and gas sparging among others. Further, a number of parameters related to nutrient consumption and metabolite production are measured off-line by periodic withdrawal of samples from the bioreactors (see the following Table 5 for examples). The parameters include physical and state parameters, chemical parameters, and physiological parameters. Due to the differences in sampling frequencies of the off-line parameters, all off-line measurements can be preprocessed using a linear interpolation method (see e.g. Charaniya, S., et al., J. Biotechnol. 147 (2010) 186-197).

TABLE 5
Overview of some conventional off-line,
at-line and in-line measured
parameters during cell cultivation.
off-line and at-line parameters
physical and state parameters
dissolved carbon dioxide
dissolved oxygen
pH (off-line)
chemical parameters
lactic acid concentration
glucose concentration
sodium ion concentration
ammonium ion concentration
osmolality
physiological parameters
viable cell density
viability
packed cell volume
integral of packed cell volume
on-line parameters
controlled parameters
dissolved oxygen (primary probe)
dissolved oxygen (secondary probe)
vessel temperature
pH (on-line)
jacket temperature
control action parameters
dissolved oxygen (Do) controller output
air sparge rate
air sparge set point
total air sparged
oxygen sparge rate
total oxygen sparged
pH controller output
total base added
CO₂ sparge rate
total CO₂ sparged
total gas sparged
others
oxygen uptake rate
reactor weight
overlay flowrate
exhaust valve pressure
backpressure

In one aspect, the invention provides a method for determining if process data acquired during the cultivation of a cell clone is affected by a problem comprising the following steps:
optionally providing a metabolic model of the mammalian or bacterial cell clone (expressing a recombinant, heterologous polypeptide),
optionally acquiring process data for a cultivation of the mammalian or bacterial cell clone expressing a recombinant, heterologous polypeptide,
fitting the process data acquired during the cultivation of the mammalian or bacterial cell clone expressing a recombinant, heterologous polypeptide using a metabolic model generated for the same mammalian or bacterial cell expressing the same recombinant, heterologous polypeptide, and
determining that the cultivation is affected by a problem when the modeled fit shows an offset with respect to the raw data of more than 10%.

In one aspect, the invention provides a method for determining if process data acquired during the cultivation of a cell clone is affected by a problem comprising the following steps:
receiving process data of the cell clone cultivation, wherein the cell clone produces a polypeptide heterologous to said cell, and
fitting the data using a metabolic model established for said cell and characterizing the fit by a statistical correlation method with a value of 1 representing a perfect fit, preferably by a chi² value determined by a Pearson's chi-squared test,
whereby the process data is affected by a problem if the correlation value (chi² value) is more than 5.

In one aspect, the invention provides a method for selecting a cell clone expressing (and producing) a heterologous polypeptide, wherein the method comprises the following steps:
a) separately cultivating a multitude of (isolated or single) cell clones that produce the same heterologous polypeptide, whereby during the cultivating temporal process data is recorded,
b) fitting the process data acquired in step a) of each clone individually using a metabolic model generated for the same cell (optionally expressing the same recombinant, heterologous polypeptide), whereby for all clones the same model is used,
c) determining that a cultivation of the multitude of cultivations of step a) is affected by a problem if the fit obtained in step b) for the process data of said cultivation obtained in step a) in said metabolic model (i) shows an offset with respect to the raw data of more than 10%, or (ii) the correlation value determined in a statistical correlation method, preferably the chi² value determined by a Pearson's chi-squared test, for the fit is 5 or more with a value of 1 being a perfect fit,
d) repeating steps a) to c) with the cell clones that had a problem in the cultivation as determined in step c), or if no clone had a problem in the cultivation as determined in step c) selecting the clone from the multitude of clones that has (i) the highest titer, and/or (ii) product quality, and/or (iii) highest score among the combination of metabolic performance indicator values (see Example 4)

In one aspect, the invention provides a method for identifying improved cultivation conditions for a cell expressing (and producing) a heterologous polypeptide, wherein the method comprises the following steps:
a) cultivating a mammalian or bacterial cell that produces or secretes a heterologous polypeptide with a first set of cultivation conditions, whereby during the cultivating temporal process data is recorded,
b) fitting the process data acquired in step a) using a metabolic model generated for the same mammalian or bacterial cell (optionally expressing the same recombinant, heterologous, produced polypeptide),
c) determining that the cultivation of step a) is affected by a problem if the fit obtained in step b) for the process data of said cultivation obtained in step a) in said metabolic model (i) shows an offset with respect to the raw data of more than 10%, or (ii) the correlation value determined in a statistical correlation method, preferably the chi² value determined by a Pearson's chi-squared test, for the fit is 5 or more with a value of 1 being a perfect fit,
d) i) repeating steps a) to c) with the first set of cultivation conditions if the cultivation had a problem as determined in step c), or ii) repeating steps a) to c) with a second set of cultivation conditions different from the first set of cultivation conditions if the cultivation had no problem as determined in step c),
e) i) repeating steps a) to d) with the same set of first cultivation conditions and a new set of second cultivation conditions that is different from any previously used set of cultivation conditions (i.e. it is different from said first and said second set of cultivation conditions and also from all other sets of cultivation conditions used in the method before) if (i) the titer, and/or (ii) product quality, and/or (iii) score of combination of metabolic performance indicator values obtained with the second set of cultivation conditions is not improved compared to the (i) the titer, and/or (ii) product quality, and/or (iii) score of combination of metabolic performance indicator values obtained with the first set of cultivation conditions, or ii) identifying the second set of cultivation conditions to be improved cultivation conditions if (i) the titer, and/or (ii) product quality, and/or (iii) score of combination of metabolic performance indicator values obtained with the second set of cultivation conditions is improved compared to the (i) the titer, and/or (ii) product quality, and/or (iii) score of combination of metabolic performance indicator values obtained with the first set of cultivation conditions.

In one embodiment of all aspects and embodiments the problem is a technical problem.

In one embodiment of all aspects and embodiments the mammalian cell or the mammalian cell clone that secretes a heterologous polypeptide has been obtained by transfecting a mammalian cell with a nucleic acid encoding the heterologous polypeptide, and expresses said heterologous polypeptide, and secretes said heterologous polypeptide into the cultivation medium.

In one embodiment of all aspects and embodiments the correlation value determined by a statistical correlation method for the fit is 2 or more. In one embodiment of all aspects and embodiments the correlation value determined by a statistical correlation method for the fit is 1 or more.

In one embodiment of all aspects and embodiments the chi² value determined by a Pearson's chi-squared test for the fit is 2 or more. In one embodiment of all aspects and embodiments the chi² value determined by a Pearson's chi-squared test for the fit is 1 or more.

In one embodiment of all aspects and embodiments the offset is an offset from the 1:1 line of modeled and measured data of more than 10%.

In one embodiment of all aspects and embodiments the mammalian cell is a CHO cell. In one preferred embodiment the CHO cell is a CHO—K1 cell.

In one embodiment of all aspects and embodiments the heterologous polypeptide is a recombinant polypeptide.

In one embodiment of all aspects and embodiments the heterologous polypeptide is a monoclonal antibody. In one embodiment the monoclonal antibody is a therapeutic monoclonal antibody.

In one embodiment of all aspects and embodiments the process data comprises the temporal values of at least 15 process parameters. In one embodiment the process data comprises the temporal values of at least 20 process parameters. In one embodiment the process data comprises the temporal values of at least 30 process parameters. In one embodiment the process data comprises the temporal values of at least 40 process parameters. In one preferred embodiment the process data comprises the temporal values of at least 12 on-line process parameters and at least 28 off-line process parameters.

In one embodiment of all aspects and embodiments the process data comprises at least 6 temporal values for each process parameter.

In one embodiment of all aspects and embodiments the metabolic model is a genome-based metabolic model. In one embodiment the genome-based metabolic model comprises five compartments. In one embodiment the five compartments are cytosol, mitochondria, endoplasmatic reticulum, Golgi apparatus and bioreactor. In one preferred embodiment the metabolic model comprises the central metabolic pathways of glycolysis, citric acid cycle, pentose phosphate pathway, and respiratory chain, the biosynthesis of the major biomass constituents' protein, lipid, RNA, DNA, and carbohydrates, C1-metabolism, and amino acid degradation pathways.

In one embodiment of all aspects and embodiments the metabolic model includes up to 1200 metabolites, up to 800 genes and up to 1500 reactions.

In one embodiment of all aspects and embodiments the metabolic model includes at least 600 reactions, 500 metabolites and 250 genes (open reading frames). In one preferred embodiment the metabolic model includes at least 654 reactions, 583 metabolites and 266 open reading frames.

In one embodiment of all aspects and embodiments the carbon balances are closed in the metabolic model. In one embodiment the closure of the carbon balance is by constraining glucose and non-essential amino acids.

In one embodiment of all aspects and embodiments the nitrogen and redox balance is closed in the metabolic model. In one embodiment the closure of the nitrogen and redox balances are by constraining ammonia production and oxygen uptake rate, respectively.

In one embodiment of all aspects and embodiments the estimation of cellular uptake and production rates is performed by first subdividing the whole fermentation process into physiologically distinct process phases (optionally through a computational optimization procedure; and/or optionally wherein the optimum number of process phases is determined using a χ²-based goodness-of-fit test). In one embodiment during each process phase, constant cell physiology is assumed and/or constant biomass-specific rates are assumed. In one embodiment biomass-specific rates are determined using nonlinear regression.

In one embodiment of all aspects and embodiments the metabolic model has been built using a four-step process comprising (i) building an initial reconstruction from gene-annotation data coupled with information from databases, which link known genes to functional categories; (ii) improving the model by using data from primary literature and converting into a mathematical model with constraint-based approaches; (iii) validating the model through comparison of model predictions to phenotypic data; and (iv) improving the metabolic reconstruction by subjecting it to continued wet- and dry-lab cycles to improve accuracy.

In one embodiment of all aspects and embodiments the metabolic model comprises only annotated open reading frames of the mammalian cell. In one embodiment the model further comprises gene products validated in literature. In one embodiment the model further comprises amino acid biosynthesis and metabolism pathways, carbohydrate biosynthesis and metabolism pathways, and nucleotide biosynthesis and metabolism pathways. In one embodiment the metabolic model further comprises transport processes. In one embodiment the metabolic model is further refined by identifying and removing repeated and/or redundant reactions.

In one embodiment of all aspects and embodiments the metabolic model is based on an average cell composition of (w/w) 74.2% protein, 1.6% DNA, 6.1% RNA, 4.5% carbohydrates, and 10.1% lipids for estimating requirements for each component in the biomass equation. In one embodiment the biomass equation further includes cholesterol.

In one embodiment of all aspects and embodiments the efficiency of oxidative phosphorylation is 2.5 expressed in the ratio of mol ATP produced per mol of electrons carried through the electron transport chain. In one embodiment the metabolic model further uses a cost in ATP for biopolymer (RNA, DNA, protein) production of 29.2 mmol ATP/g dry weight. In one embodiment the metabolic model further uses a value of 1.55 mmol ATP/g DW/h for maintenance and of 37.8 mmol ATP/g DW/h for ATP yield and of 8.6 mmol ATP/g DW/h for growth-associated maintenance.

In one embodiment of all aspects and embodiments the metabolic model comprises the rates for uptake, metabolism and secretion rates of essential amino acids, folate and phosphate. In one embodiment the metabolic model further comprises uptake, metabolism and secretion rates of biotin, thiamine, vitamins, calcium and magnesium ions.

In one embodiment of all aspects and embodiments the uptake of glucose, oxygen, and glutamine are fixed at the experimentally observed rates in the metabolic model. In one embodiment further the lactate, ammonia, glutamate, aspartate, and alanine uptake, metabolism, and secretion rates are left unconstrained in the metabolic model.

In one embodiment of all aspects and embodiments the uptake rates for essential amino acids are removed in the metabolic model. In one embodiment further the uptake or production rates for non-essential amino acids (preferably for serine, asparagine, glycine, and proline) are fixed at the experimentally observed rates in the metabolic model.

In one embodiment of all aspects and embodiments the metabolic model combines genetic and signaling regulatory elements, enzyme kinetics and chemico-physical parameters in hybrid model approaches.

In one embodiment of all aspects and embodiments the maximum production rate of the monoclonal antibody is set to a value of 0.0084 mmol antibody/g DW/h (DW=dry weight).

In one embodiment of all aspects and embodiments metabolic fluxes for the metabolic model are determined by constraints-based flux analysis of the metabolic network model subjected to stoichiometric (metabolite mass balance) and thermodynamic (reaction reversibility) constraints.

In one embodiment of all aspects and embodiments the metabolic model comprises three distinct phases. In one preferred embodiment the three phases are (i) an initial exponential growth phase lasting for day 1 to day 3; (ii) a late exponential growth phase lasting from day 4 to day 6; and (iii) an early stationary phase lasting from day 8 to day 10.

In one embodiment of all aspects and embodiments the cellular objective in the first phase of the metabolic model is biomass production (and this is to be maximized).

In one embodiment of all aspects and embodiments the cellular objective in the second phase of the metabolic model is energy optimization (and is to be minimized).

In one embodiment of all aspects and embodiments the cellular objective in the third phase of the metabolic model is protein production (and this is to be maximized).

In one preferred embodiment of all aspects and embodiments the cellular objective in the first phase of the metabolic model is biomass production (and this is to be maximized), the cellular objective in the second phase of the metabolic model is energy optimization (and is to be minimized), and the cellular objective in the third phase of the metabolic model is protein production (and this is to be maximized).

In one embodiment of all aspects and embodiments metabolic network models and hybrid models thereof is used for any kind of cell cultivation strategies like batch, split-batch, fed-batch, perfusion, intensified and continuous cultivations for (i) simulating uptake and consumption rates, (ii) simulating intracellular fluxes and concentrations and (iii) check for data consistency, accuracy, and completeness.

In one embodiment of all aspects and embodiments the metabolic model is checked during the reconstruction process iteratively for consistency, accuracy, and completeness by comparing simulated results with experimental results and adopted/adjusted until simulated results are within 10% of the experimental results (optionally both quantitatively and qualitatively).

The goodness-of-fit of a statistical model can be used to characterize the quality of a model with respect to the underlying modeled process, i.e. how good the correlation between model and experimental data is. Generally, the goodness-of-fit sums up the deviations between experimental values and the values predicted by the model.

One way to describe the goodness-of-fit is the Pearson's chi-squared test. It is based on the sum of differences between experimental and modeled outcome frequencies, each squared and divided by the expectation:

$${χ}^{2} = {\sum }_{i = 1}^{n} \frac{{( {O}_{i} - {E}_{i} )}^{2}}{{E}_{i}}$$

wherein
Oᵢ denotes an experimentally determined frequency (i.e. count) for bin i
Eᵢ denotes a modeled frequency for bin i, asserted by the null hypothesis.

Eᵢ can be calculated by:

Eᵢ=(F(Yᵤ)−F(Yₗ))N

wherein
F denotes the cumulative distribution function of the distribution being analyzed
Yᵤ denotes the upper limit for class i,
Yₗ denotes the lower limit for class i, and
N denotes the number of data points.

The obtained value can be compared with a chi-squared distribution in order to determine the goodness of fit.

On average it can be expected that each term is about 1. Therefore, it is likewise expected that the total should correlate to the number of data points. The number of data points that could not be automatically hit by the fitted function is called the number of “degrees of freedom”.

What is decisive is the relative size of the deviation and the error bar. “Good” points have a small (less than 1) ratio of deviation (Δ) to error (σ); “Bad” points have a ratio of deviation to error larger than one, and hence the curve fails to go through the error bar (as in the third data point). On average a good fit will have as many unusually large deviations as unusually small deviations, that is, on average the ratio of deviation to error will be about 1. (Of course, in a perfect fit the curve will go right through every data point: zero deviation.) χ² is defined as the sum of the square of each data point's ratio of deviation to error:

$${χ}^{2} = {( \frac{{Δ}_{l}}{{σ}_{1}} )}^{2} + {( \frac{{Δ}_{2}}{{σ}_{2}} )}^{2} + {( \frac{{Δ}_{3}}{{σ}_{3}} )}^{2} + … + {( \frac{{Δ}_{N}}{{σ}_{N}} )}^{2}$$

The degrees of freedom (d.f.) equals the number of data points reduced by the number of adjustable parameters.

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1A and FIG. 1B Metabolic model fit of a 14 day fed-batch cultivation experiment with corrupted and corrected feed concentration data. The 14 day fed-batch cultivation is divided into 5 different metabolic phased (horizontal lines) based on the discrete measured in-process data (black boxes with generic error variances). The black line fits the rates of consumed of produced metabolite (based on drifting amounts). The offset of measured and modeled data (A) indicate corrupted data inputs. The match of measured and modeled data is shown for a corrected data set (B).

FIG. 2 Correlation plot of mean and standard deviation from rates determined from measured amounts plotted against reconciled model rates. Shown are fermentations 9 (light-grey circles), 14 (grey inverted triangles) and 51 (black squares). The dashed line denotes the 1:1 correlation line. The rates are determined from measured amounts.

FIG. 3A and FIG. 3B χ² values of the different cultivations and model scenario combination. The χ² displayed is the median of χ² in each metabolic phase and model variants (model 1 to model 6) for tested fermentation batches (see also Table 3). The number to the right of the heat map is the replicate group.

FIG. 4A and FIG. 4B Viable cell density and lactate kinetics of two different recombinant CHO clones (clone 1 and clone 2), expressing the same product are shown. The cells were cultivated by a fed-batch process and analyzed by discrete at-line in process control analytics.

FIG. 5 Modeled rates of recombinant CHO clone 1 and clone 2, expressing the same product in a 14 day fed-batch process. Measured (reconciled) rates (black boxes with generic error variance) and modeled (black box) rates are shown for all five metabolic phases (horizontal lines).

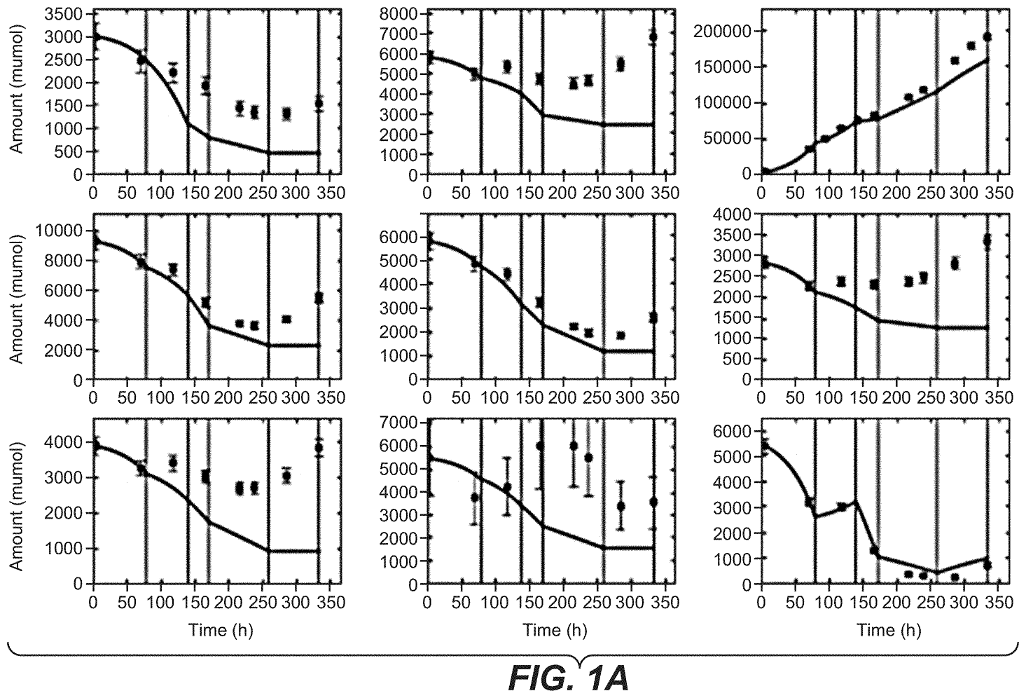

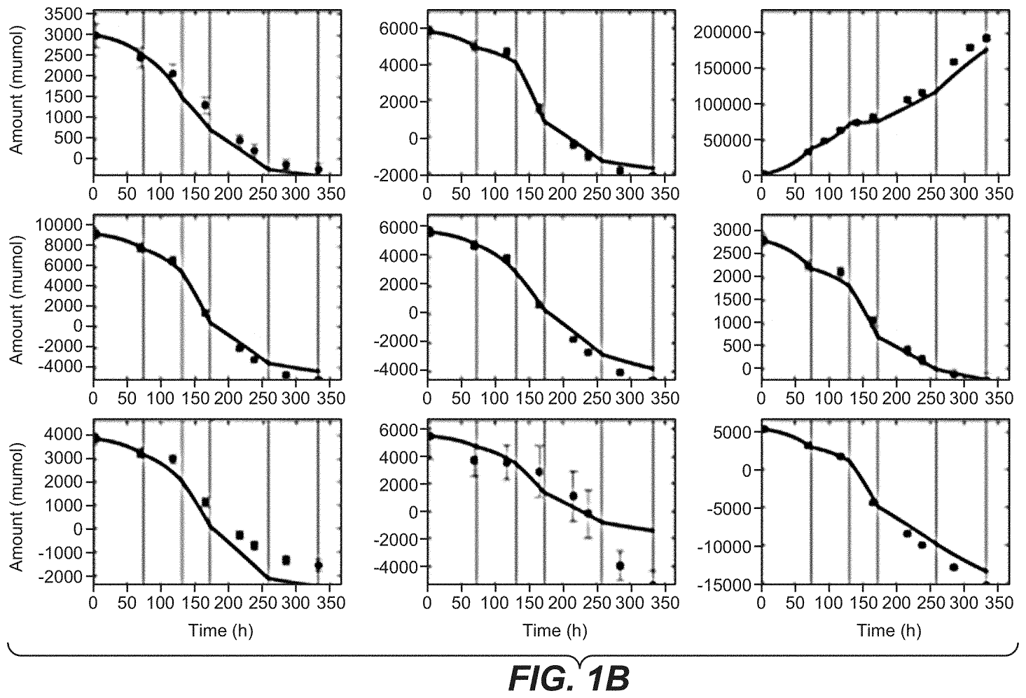

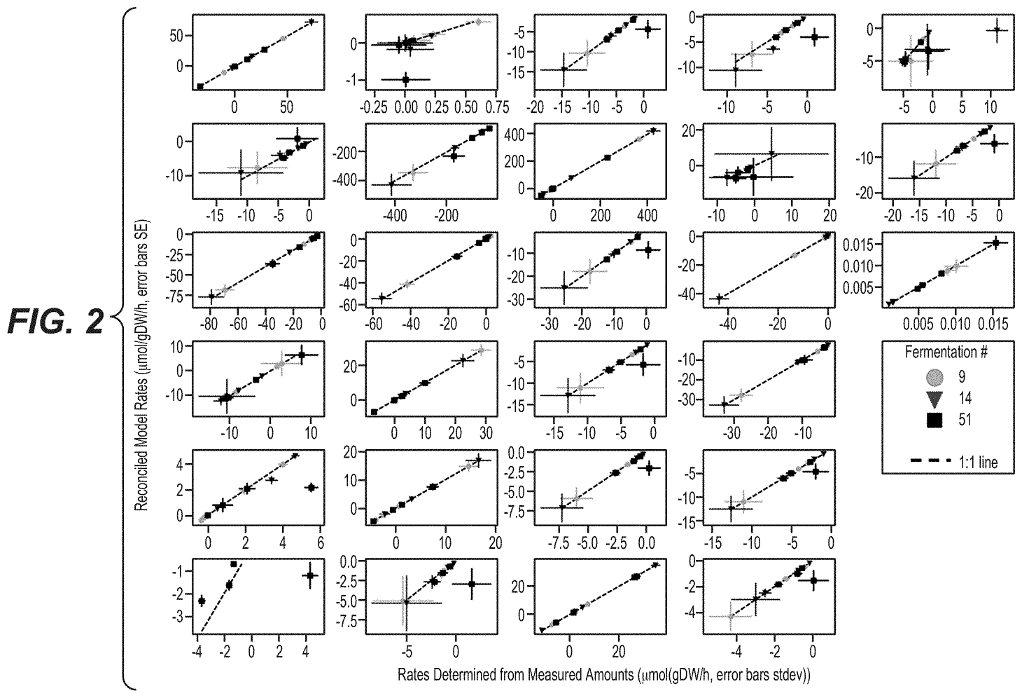

### EXAMPLES

Materials and Methods

Product Quantification, Metabolite and Amino Acid Analysis:

For the quantification of product titer, metabolite and amino acid concentrations in fermentation broth cells were removed by centrifugation. Glucose, lactate, and ammonium concentrations were measured using a Cedex Bio HT bioprocess analyzer (Roche Diagnostics GmbH, Mannheim) using specific assays. Cell-free supernatant was sterile filtered by 0.2 μm or 3 kDa membrane for subsequent protein quantification or amino acid analysis, respectively. Product titers were quantified by a Poros A HPLC method as described previously [Zeck et al., 2012]. Amino acid levels in fermentation supernatant were measured by an in-house method using an Agilent RRLC 1200 system (Agilent Technologies, Santa Clara) and a fluorescence detector.

Protein Determination:

For the extraction of cellular protein content, the Mem-PER™ Plus Membrane Protein Extraction kit (Thermo Scientific, Darmstadt) and the Cedex HiRes analyzer were applied. In the first step, a specific amount of living cells was collected using the Cedex HiRes analyzer and transferred into a falcon tube. The cellular proteins were then extracted according to protocol 2 of the enclosed Mem-PER™ instruction sheet for suspension mammalian cells (Instructions Manual No. 89842, Thermo Scientific, Darmstadt). After the proteins were extracted and collected in a 1.5 mL tube, the protein concentration was measured using the Bradford Coomassie® Plus™ assay kit and the microplate procedure A (Instructions Manual No. 23236, Thermo Scientific, Darmstadt). In this case, a proprietary CHO host cell protein standard, instead of the normal BSA protein standard was used to take advantage of the equity between the measured CHO proteins of a given sample and the standard curve made out of the proprietary host cell protein mixture.

Afterwards, the measured protein content c_{Protein, measured} is combined with the total cell density TCD and viability V data from the Cedex HiRes analyzer and of course with the volume of the test tube V_{Protein,tube} and the cell containing sample volume Vₛₐₘₚₗₑ, thus the protein content per cell is calculated as follows:

$${c}_{P r o t e i n / Cell} = \frac{{c}_{Protein , measured} \cdot {V}_{P r o t ein , tube}}{TCD \cdot V \cdot {V}_{Sample}}$$

Determination of Average Single Cell Mass, Volume and Density:

For the determination of average cell mass, the Cedex HiRes Analyzer (Roche Diagnostics GmbH, Mannheim, Germany) machine is used in the first place to determine the cell concentration of a given sample. Moreover, the Cedex HiRes device provides morphological parameters like cell diameter (used to calculate the cell volume within the device), cell viability and aggregation rates.

The cell mass determination is based on the assumption, that the whole cell mass m_{Cell,Total} consists of the sum of cellular biomass m_{Cellular biomass} (cell membrane, cell components, proteins, e.g.) and water m_{Water}.

m_{Cell,Total}=m_{Cellular biomass}+m_{Water}

A cell containing sample was pipetted in a balanced falcon tube and separated from the supernatant. A wash step ensures, that only cells are left in the falcon. Afterwards the falcon was dried in a dry cabinet at 80° C. for at least 24 hrs. in order to eliminate the water. Thus, the cellular biomass can be measured by the weight difference of an empty falcon tube m_{Falcon,empty} and a falcon tube with dried biomass m_{Falcon,dried.} Combined with the measured total cell density TCD and the sample volume V_{Sample}, the average cell mass can be calculated as follows:

$${m}_{Cell , Total} = {m}_{Cellular biomass} = \frac{{m}_{Falcon , dried} - {m}_{Falcon , empty}}{TCD \cdot {V}_{sample}}$$

Determination of Oxygen Uptake Rate (OUR):

For the determination of the oxygen uptake rate OUR, the dynamic method was applied. The dynamic method is a well-known standard procedure and is generally based on the oxygen consumption of a submerged cell culture. During fermentation, the dissolved oxygen concentration (measured by a Clark electrode) inside the bioreactor is regulated to a defined value and therefore the temporal change of dissolved oxygen can be considered as 0.

$$\frac{{dc}_{{0}_{2}}}{{d}_{t}} \approx 0$$

For application of the dynamic method, the gassing is interrupted for a certain time resulting in decrease of dissolved oxygen only by respiratory activity of the cells which can be recorded by the oxygen probe.

$$\frac{{dc}_{{0}_{2}}}{{d}_{t}} = - OUR$$

OUR can be determined by the depletion of dissolved oxygen until the gassing is reactivated.

CHO Network Model Reconstruction and Flux Analysis:

A genome-based CHO network model comprising five compartments (cytosol, mitochondria, ER, Golgi, bioreactor) was constructed from public sources including databases and primary literature, according to established procedures [Sheikh et al., Biotechnol. Prog. 21 (2005) 112-121; Selvarasu et al., Mol. Biosyst. 6 (2010) 152-161; Oberhardt et al., Mol. Syst. Biol. 5 (2009) 320]. In addition to central metabolic pathways (glycolysis, citric acid cycle, pentose phosphate pathway, respiratory chain), the model describes biosynthesis of major biomass constituents (protein, lipid, RNA, DNA, carbohydrates), C1-metabolism, and amino acid degradation pathways. For the recombinant protein product investigated, a corresponding set of product formation reactions was formulated, which accounted for the amino acid composition and a representative glycosylation structure (two sialilated biantennary glycans per product molecule) of the recombinant protein. The resulting model comprised 654 reactions, 583 metabolites, and represented 266 ORFs. The biomass composition was chosen comparable to previous studies for CHO cells or murine cell lines (see e.g. Altamirano et al., Biotechnol. Prog. 17 (2001) 1032-1041; Bonarius et al., Biotechnol. Bioeng. 50 (1996) 299-318; Selvarasu et al., Biotechnol. Bioeng. 109 (2012) 1415-1429). See the following Tables 6 and 7.

TABLE 6
Biomass composition of CHO-K1 cells employed in
network simulations.

| | content per | mass | |
|---|---|---|---|
| | average | fraction | |
| compound | cell [pg] | [% w/w] | remark |
| protein | 195 | 40.8 | Popp, O., et al. |
| | | | (Biotechnol. Bioeng. 113 |
| | | | (2016) 2005-2019) |
| RNA | 38 | 7.9 | acc. to Table 2 of Hu W—S, |
| | | | Zhou W, editors. 2012. |
| | | | Cell culture bioprocess |
| | | | engineering. Minnesota, |
| | | | Minn. University and |
| | | | scaled to 100% |
| DNA | 10 | 2.0 | acc. to Table 2 of Hu W—S, |
| | | | Zhou W, editors. 2012. |
| | | | Cell culture bioprocess |
| | | | engineering. Minnesota, |
| | | | Minn. University and |
| | | | scaled to 100% |
| Lipid | 96 | 20.0 | acc. to Table 2 of Hu W—S, |
| | | | Zhou W, editors. 2012. |
| | | | Cell culture bioprocess |
| | | | engineering. Minnesota, |
| | | | Minn. University and |
| | | | scaled to 100% |
| carbo- | 124 | 25.8 | acc. to Table 2 of Hu W—S, |
| hydrates | | | Zhou W, editors. 2012. |
| | | | Cell culture bioprocess |
| | | | engineering. Minnesota, |
| | | | Minn. University and |
| | | | scaled to 100% |
| potassium | 17 | 3.5 | as main representative of |
| | | | ash content [Alberts B, et |
| | | | al., 1994. Molecular |
| | | | Biology of the Cell, |
| | | | Garland Science, p. 508; |
| | | | ash content acc. to |
| | | | [Vriezen, 1998, |
| | | | Physiology of Mammalian |
| | | | Cells in Suspension |
| | | | Culture, PhD Thesis, TU |
| | | | Delft.] |

| | | |
|---|---|---|
| avg. single- | 479 pg DW/cell | Popp, O., et al. |
| cell mass | | (Biotechnol. Bioeng. 113 |
| | | (2016) 2005-2019) |
| avg. cell | 1,530 fL | Popp, O., et al. |
| volume | | (Biotechnol. Bioeng. 113 |
| | | (2016) 2005-2019) |
| avg. single- | 313 pg DW/L_{cell} | calculated from cell |
| cell density | | volume and avg. cell mass |
| | | above |

TABLE 7
Macromolecule composition of CHO-K1 cells employed in network
simulations.

| | Remark | Mol-% | Reference |
|---|---|---|---|
| DNA | GC content | 42 | [Mouse |
| | | | Genome |
| | | | Sequencing |
| | | | Consortium, |
| | | | 2002] |
| RNA | each of A, C, G, and U | 25 | |
| protein | ala | 6.1 | Mouse |
| | arg | 5.7 | Proteome |
| | asn | 3.6 | (originally |
| | asp | 4.8 | available from |
| | cys | 2.4 | [European |
| | gln | 4.7 | Bioinformatics |
| | glu | 6.8 | Institute, |
| | gly | 6.6 | 2003]) |
| | his | 2.6 | |
| | ile | 4.5 | |
| | leu | 9.9 | |
| | lys | 5.7 | |
| | met | 2.3 | |
| | phe | 3.9 | |
| | pro | 6.2 | |
| | ser | 8.5 | |
| | thr | 5.4 | |
| | trp | 1.3 | |
| | tyr | 2.8 | |
| | val | 6.2 | |
| lipids | cholesterol | 10.4 | [Cadigan et al, |
| | cholesterol esters | 5.7 | 1988, J. Biol. |
| | cardiolipine | 0.8 | Chem. 263: |
| | phosphatidic acid | 0.8 | 274-282.; |
| | phosphatidylcholine | 49.5 | Emoto et al., |
| | phosphatidylethanolamine | 13.5 | 1999, Proc. |
| | phosphatidylglycerol | 0.8 | Natl. Acad. |
| | phosphatidylinositol | 4.9 | Sci. USA 96: |
| | phosphatidylserine | 4.7 | 12400-12405.; |
| | sphingomyelin | 8.6 | Brasaemle et |
| | triacylglycerol | 0.3 | al., 2000, J. |
| | | | Biol. Chem. |
| | | | 275: 38486- |
| | | | 38493] |
| carbohydrates | synthesized by polymerization | | |
| | of UDP-glucose | | |

Model reconstruction and model simulations were performed using Insilico Discovery802™ software v 3.2 (Insilico Biotechnology AG, Stuttgart). For model verification, it was confirmed that the elemental balance and charge balance closed for all reactions. Moreover, Flux Balance Analysis [Savinell and Paulson, J. Theor. Biol. 154 (1992) 421-454 and 455-473] was used to verify functionality of individual pathways. Time-series transcript data collected during CHO fermentations served to delineate (in)active metabolic routes in the network and supported identification of predominant isoenzyme species. The resulting network model still contained inner degrees of freedom that cannot be resolved from measurement of extracellular metabolites and network stoichiometry alone. In such cases, the most energetically efficient metabolic route was considered active and the others inactive in order to ensure comparability of flux distributions obtained. Importantly, this choice did not affect the reconciled values of cellular uptake and production rates inferred from measurements.

Estimation of cellular uptake and production rates was performed by first subdividing the whole fermentation process into physiologically distinct process phases through a computational optimization procedure. This optimization was performed employing an evolutionary strategy [Müller et al., 2009, Proceedings of the 11th Annual conference on Genetic and evolutionary computation. Montréal, Canada: ACM pp. 1411-1418. Available: http://dl.acm.org/citation.cfm?id=1570090] using the measured time-series data of cell number, protein product, and extracellular metabolites as input. The optimum number of process phases was determined using a χ²-based goodness-of-fit test akin to the method reported by Leighty and Antoniewicz [Metab. Eng. 13 (2011) 745-755]. During each process phase, constant cell physiology was assumed, implying constant biomass-specific rates. These biomass-specific rates were determined using nonlinear regression. Uptake rates of individual nutrients were corrected for the influence of chemical decomposition based on half-life data determined from control fermentations performed without inoculation where needed. The resulting uptake and production rates served as inputs for performing Metabolic Flux Analysis [Maier et al., Biotechnol. Bioeng. 100 (2008) 355-370; Niklas et al., Curr. Opin. Biotechnol. 21 (2010) 63-69; Stephanopoulos et al., 1998, Metabolic engineering: Principles and methodologies. San Diego: Academic Press] and thermodynamic consistency of the computed flux distributions was confirmed (i.e. no violations of directionality for known irreversible reactions).

Definition of Performance Measures and Multivariate Data Analysis:

Computation of Pearson and Spearman correlations of metabolite data, process data, and of intracellular flux distributions were performed using the statistical software R v2.13.2, the Stats package [R Core Team, 2013], JMP (SAS, Marlow), and Qlucore (Qlucore AB, Lund). Correction of p values for multiple testing was performed using the method of Benjamini and Hochberg [Benjamini and Hochberg, 1995] for controlling the False Discovery Rate (FDR) at FDR<0.05 or as indicated. Composite selection scores for each cultivation were calculated as follows: a composite score CS was defined as weighted sum of category scores (CASᵢ)

$$C S = {\sum }_{i = 1}^{{n}_{categories}} {w}_{i} \cdot {CAS}_{i}$$

where wᵢ ∈ [0,1] and Σᵢ₋₁ⁿ^{_{categories}} wᵢ=1. Weighting factors were chosen as appropriate for a given selection process. Each category score CASᵢ was specified as weighted average of the individual indicators contained in the category (IND_{i,j})

$$C A {S}_{i} = {\sum }_{j = 1}^{{n}_{IND , i}} {w}_{i , j}^{IND} \cdot {IND}_{i , j}$$

again with w_{i,j}^{IND} ∈ [0,1] and Σⱼ₌₁ⁿ^{_{IND,i}}w_{i,j}^{IND}=1. Each indicator IND_{i,j} was determined as scaled and time-weighted average of a given performance measure PM^{scaled}:

$${IND}_{i , j} = {\sum }_{k = 1}^{{n}_{time points}} {w}_{k}^{IND , j} \cdot {PM}_{i , j}^{scaled} ( {t}_{k} )$$

with the same restrictions applying to the wₖ^{INDi,j} as described for the weighting factors above and PM_{i,j}^{scaled}(tₖ)≥0.

Here, concentrations, molar amounts, biomass-specific uptake/production rates, intracellular fluxes or ratios were employed as performance measures, which were normalized to non-negative dimensionless quantities using a suitable reference value. Different scaling procedures can be employed to achieve these properties and to distinguish between properties where a high value is considered desirable (e.g. product titer) and those where low values are preferred (e.g. byproduct yield). Herein the range of observed values for scaling used was as follows

$${PM}_{i , j}^{scaled} \{ \begin{matrix}\begin{matrix}\frac{{PM}_{i , j} ( {t}_{k} ) - {PM}_{i , j}^{min}}{{PM}_{i , j}^{max} - {PM}_{i , j}^{min}} & if high value of {PM}_{i , j} is better \\ \frac{{PM}_{i , j}^{max} - {PM}_{i , j} ( {t}_{k} )}{{PM}_{i , j}^{max} - {PM}_{i , j}^{min}} & if low value of {PM}_{i , j} is better\end{matrix} & \end{matrix}$$

Performance measures PM_{i,j} were defined such that they assumed only non-negative values and the PM_{i,j}^{max} and PM_{i,j}^{min} represent the maximum and minimum values of the performance measure over all clones and time points, respectively. With this choice of scaled performance indicators and weighting factors, attainable values for the composite score CS fall into the range between 0 and 1. The latter value would be assumed only if one clone exhibited the maximum observed indicator value for every indicator and for all time points where this indicator receives a non-zero weight.

Exemplary Calculation of the Titer Score of a Single Clone

When considering final titer, the Product Formation category score was set to W_{ProductFormation}=1 and wᵢ=0 for all other categories i. Since product titer is the sole active criterion its weighting within the ProductFormation category would be w_{ProductFormation,Max Titer in Metabolic Phase}^{IND}=1 whereas w_{ProductFormation,Specific Productivity}^{IND}=0 and w_{ProductFormation,Product Titer increase in Metabolic Phase}^{IND}=0. For the scaled performance measure, it is

$${IND}_{i , j} = {\sum }_{k = 1}^{{n}_{time points}} {w}_{k}^{INDi , j} \cdot {PM}_{i , j}^{scaled} ( {t}_{k} )$$

with i=“Product Formation” and j=“Max. Titer in Metabolic Phase”. Since a high titer value is preferred, the scaled performance measure is computed from

$$P {M}_{i , j}^{scaled} ( {t}_{k} ) = \frac{P {M}_{i , j} ( {t}_{k} ) - P {M}_{i , j}^{min}}{P {M}_{i , j}^{max} - P {M}_{i , j}^{min}}$$

For an exemplary clone these values are shown in the following Table 8.

TABLE 8
Example calculation of a final titer score according to the weighting procedure outlined herein.

| Time tₖ | t₁ = 0 h | t₂ = 77 h | t₃ = 154 h | t₄ = 231 h | t₅ = 308 h |
|---|---|---|---|---|---|
| Process Phase | 1 | 2 | 3 | 4 | 5 |
| wₖ^{INDi,j} | 0 | 0 | 0 | 0 | 1 |
| PM_{i,j} | 0.02 g/L | 0.09 g/L | 0.50 g/L | 1.57 g/L | 2.55 g/L |
| PM_{i,j}^{scaled} | $( 0.007 = \frac{0.02 \frac{g}{L} - 0.0 \frac{g}{L}}{2.81 \frac{g}{L} - 0.0 \frac{g}{L}} )$ | 0.032 | 0.178 | 0.559 | 0.907 |
| wₖ^{INDi,j} * PM_{i,j}^{scaled}(tₖ) | 0 | 0 | 0 | 0 | 0.907 |

| | |
|---|---|
| IND_{i,j} | 0.907 |

### Example 1

Comparison of Recombinant CHO Clones and Ranking by Metabolic Performance Indicators

For clone comparison experiments, ten recombinant CHO—K1 clones (CL4 to CL13) expressing the same monoclonal IgG4 antibody were used. For comparison studies of different products, a further clone CL14 expressing the same recombinant human IgG4 monoclonal antibody as described before and two other production clones (CL2 and CL3) expressing a monoclonal IgG1 antibody were used. The recombinant CHO—K1 clones were cultivated in a protein-free, chemically-defined proprietary medium for seed train and subsequent fed-batch experiments. Seed train cultivation was performed in shake flasks using a humidified incubator with set point controlled 7% CO₂ and 37° C. The clones were split every three to four days. For all experiments, clones of identical age in culture (21 days) until start of the experiments were used. For the fed-batch clone comparison experiments CL4 to CL13 were cultivated in 230 mL medium in 500 mL shake flasks for 13 days using a protein-free and chemically-defined proprietary base media. Two protein-free and chemically-defined proprietary feed media (feed A and feed B) were supplemented daily from day 3 (feed A, 3% of start cultivation volume per day) or day 6 (feed B, 2% of start cultivation volume per day) onwards. Fully controlled clone cultivation fed-batch experiments were performed in 2 L small-scale bioreactors (Sartorius Stedim, Göttingen) for 14 days using the same protein-free, chemically-defined proprietary base and feed media as described before. For the production-like process the same two protein-free and chemically-defined proprietary feed media (feed A and feed B) were supplemented daily from day 3 (feed A, 2% of start cultivation volume per day) or day 6 (feed B, 2% of start cultivation volume per day) until day 14. All cultivation experiments were run in triplicate. For analysis of viable and total cell densities and cell diameter an automated Cedex HiRes system (Roche Diagnostics GmbH, Mannheim) was used. Viable and total cell densities were discriminated using the trypan blue exclusion staining method according to the manufacturer's specifications. Product titer, metabolite, and amino acid concentrations in fermentation broth were quantified as described previously (Zeck et al., 2012).

For assessment of the metabolic fingerprint and overall performance of recombinant CHO clones, a metabolic flux model was used to calculate predefined metabolic performance indicators (see Table 2) and a respective scoring system to generate an aggregated and cumulative value (see Table 8). By that, allowing an automatic and user independent (avoiding individual interpretations) generation of clone rankings for CHO clone development and selection.

### Example 2

Comparison of Different Fermentation Scales for Recombinant CHO Clone Cultivations

A metabolic flux analysis approach was applied for establishing an automated CHO cell performance analysis for high throughput use. To this end, a rich data set compromising cultivations conducted at various scales, expressing various monoclonal antibodies was utilized and curated, if required (see Table 3). Methods used to design the pipeline included genome-scale metabolic network modeling, identification of process phases, metabolic flux analysis, and analysis of clone performance indicators. Statistical analyses performed included reduced χ² tests, cross-validation and replicate analyses. Results of the analyses enabled to resolve conversion and transformation errors in the data set, determine an acceptance window for the χ² tests. Further, the impact of taking into account additional measurement parameters in the form of host cell protein and oxygen uptake measurements was analyzed.

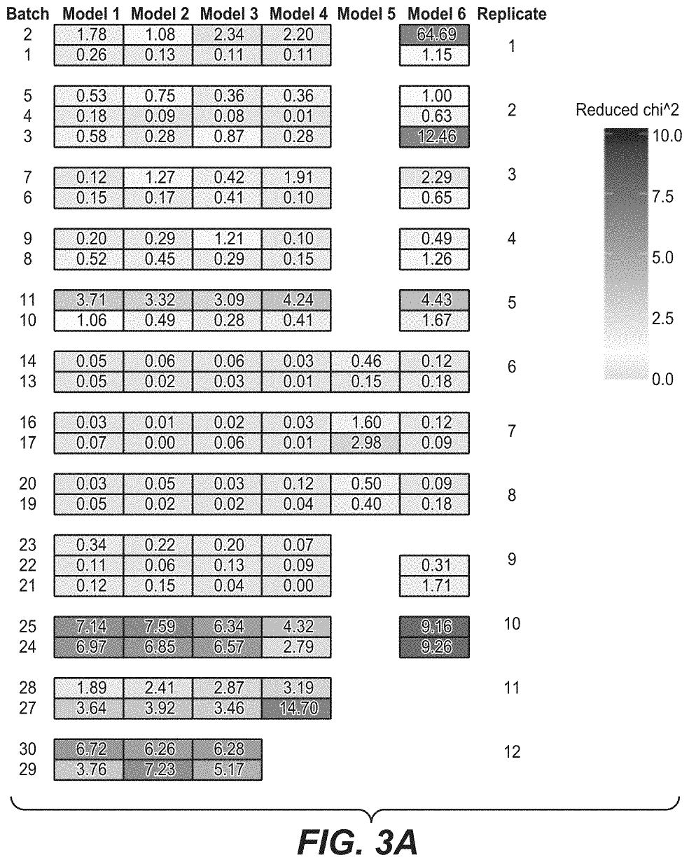

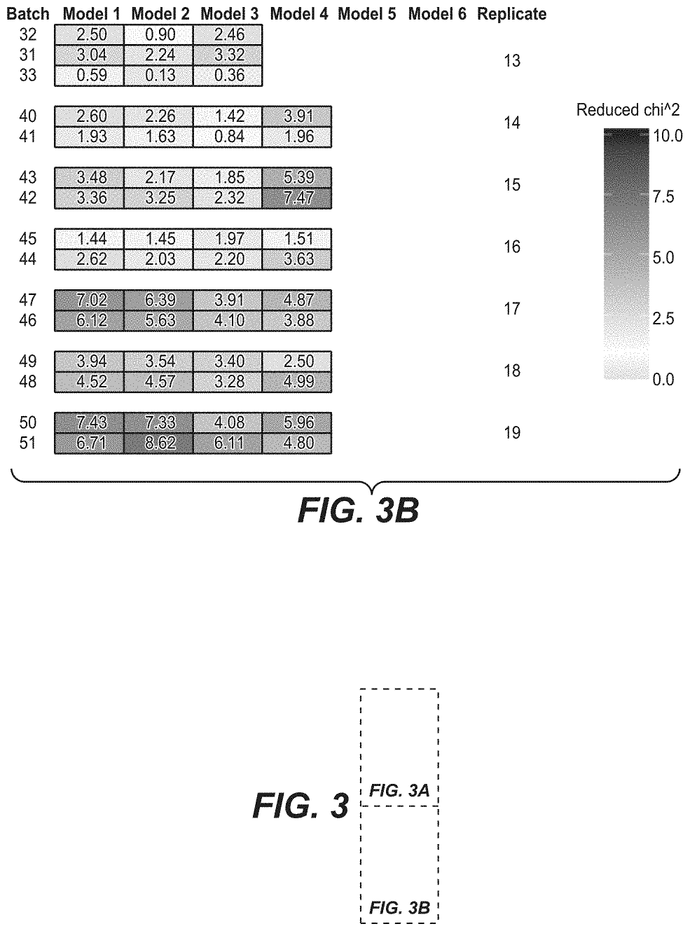

In the initial phase a consolidated data basis for metabolic analysis was established. A model scenario-based approach for testing the influence of different assumptions in the model setup (different cell biomass composition) and of inclusion of data on the reduced χ² and performance indicator values were analyzed using fed-batch cultivation data together with comprehensive cell analysis data and elemental analysis from CHO processes of different scales, carried out with different cell lines, clones, products or platforms. The different assumptions and parameters resulted in Models 1 to 6 (see Table 4 and FIG. 3).

### Example 3

CHO Clone Performance Analysis by OUR and HCP Measurement

The impact of including the additional measurements, to this end, subsets of cultivations representing the HCP and OUR data sets were analyzed (see Table 3). Here, it has been found that in both cases, taking into account the additional data improved the information content retrieved from the experiment by increasing the χ². In detail, a significant increase in the information content of CHO fermentations were gained, when taking into account host cell protein (13% points increase) and oxygen uptake rate (25% points increase) measurements (see Table 9).

TABLE 9
Detailed outcome of the χ² test. For the analysis of the impact of the HCP
and OUR measurement model scenario 1 (Model 1) was re-evaluated
using only the cultivations in which HCP and OUR were measured.

| | | consistent | | | no |
|---|---|---|---|---|---|
| | | and enough | limited | data | eval- |
| | | informa- | infor- | inconsis- | uation |
| cultivations scenario | | tion | mation | tent | possible |
| all | Model 1 | 16% | 39% | 45% | 0% |
| cultivations | Model 2 | 23% | 39% | 35% | 4% |
| | Model 3 | 21% | 42% | 37% | 0% |
| | Model 4 | 13% | 48% | 40% | 0% |
| HCP | Model 1 | 17% | 63% | 20% | 0% |
| measurements | Model 1 | 30% | 38% | 32% | 0% |
| | with | | | | |
| | HCP | | | | |
| | module | | | | |
| OUR | Model 1 | 4% | 96% | 0% | 0% |
| measurements | Model 1 | 29% | 50% | 21% | 0% |
| | with | | | | |
| | OUR | | | | |
| | module | | | | |

### Example 4

Analysis Metabolic Phenotypes of CHO Clones with Different Lactate Metabotype

Lactate is the most prominent by-product of a CHO cultivation and, by that, the concentration level in the cultivation broth and the cell specific formation and consumption rates are routinely analyzed as fermentation in process control analysis. Final candidates of a CHO clone development evaluation process often origins from the same or related CHO parental cells and/or pools. Yet, the metabotype—the metabolic phenotype in a culture—can differ immense.

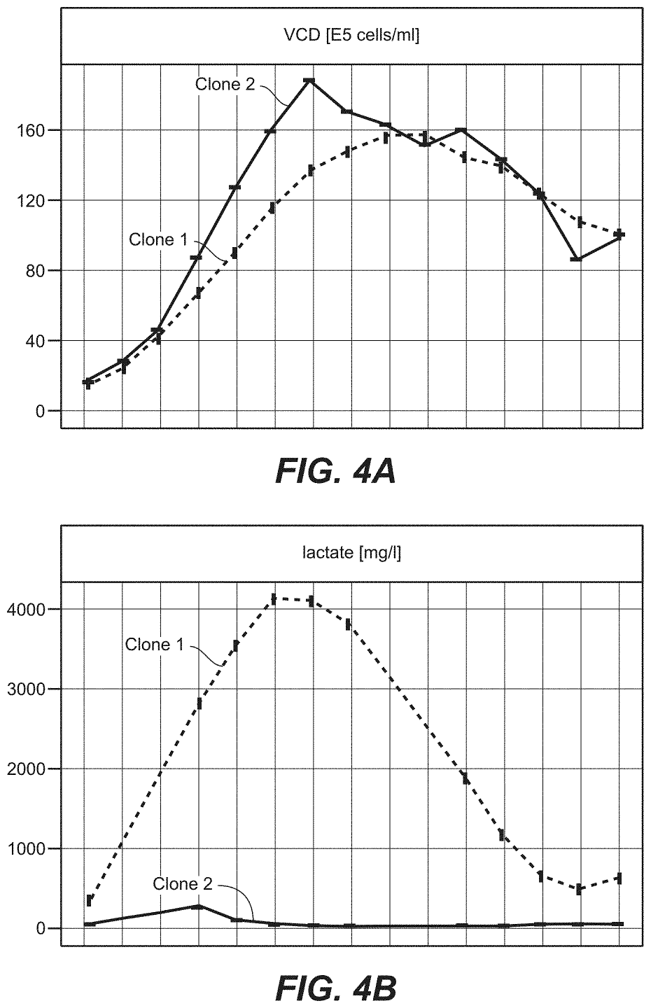

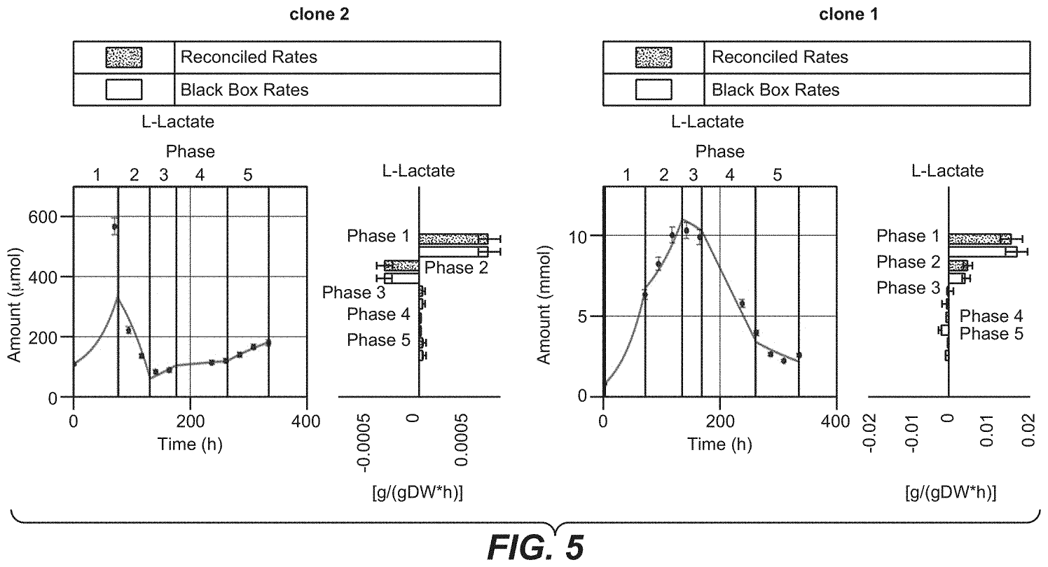

In a clone evaluation process, two different CHO clones expressing the same recombinant product were evaluated in a fed-batch experiment. By that, the cell growth of clone 1 and clone 2 vary only marginally, however, the measured lactate concentrations showed substantial differences (FIG. 4A and FIG. 4B). Clone 1 reached maximum lactate levels of 4000 mg/L in the middle of the cultivation with a subsequent remetabolization phenotype. In contrast, clone 2 did not even reach 500 mg/L lactate in maximum and the overall level in most of the process time was not measurable. To evaluate if this lactate measurements of clone 2 origins form a true metabotype or from e.g. a wrong measurement, corrupted data, etc., a metabolic flux analysis approach according to the current invention was used to analyze the probability of the lactate values. For that, the model considers lactate and all measured in-process control parameters beside lactate. The match of the reconciled lactate rates and the modeled “black box” rates for all identified five metabolic phases of clone 1 and clone 2 confirmed the correctness clone 2 lactate metabotype (FIG. 5).

### CITED DOCUMENTS (ALL INCORPORATED BY REFERENCE HEREIN)

Alberts B, et al., 1994, Molecular Biology of the Cell, Garland Science.

Altamirano C, et al., 2001, Biotechnol Prog 17: 1032-1041.

Bareither R, Pollar D, 2011, Biotechnol Prog 27: 2-14.

Benjamini Y, Hochberg Y, 1995, J R Stat Soc. B 57: 289-300.

Birzele F, et al., 2010, Nucleic Acids Res 38: 3999-4010.

Bonarius H P, et al., 1996, Biotechnol Bioeng 50: 299-318.

Brasaemle D L, Perilipin A, 2000, J Biol. Chem. 275: 38486-38493.

Brinkrolf K, et al., 2013, Nat Biotechnol 31: 694-695.

Cadigan K M, et al., 1988, J Biol Chem 263: 274-282.

Carrillo-Cocom L M, et al., 2015, Cytotechnology, 67: 809-820.

Charaniya, S., et al., J. Biotechnol. 147 (2010) 186-197.

Chen N, et al., 2012, Curr Opin Biotechnol 23: 77-82.

Chong L, et al., 2013, J Biotechnol 165: 133-137.

DeMaria C T, et al., 2007, Biotechnol Prog 23: 465-472.

Dietmair S, et al., 2012, Bioeng 109: 1404-1414.

Dietmair S, et al., 2012, PLoS ONE 7: e43394.

Emoto K, et al., 1999, Proc Natl Acad Sci USA 96: 12400-12405.

European Bioinformatics Institute. Mouse Amino Acid Composition, available: http://www.ebi.ac.uk/proteome/MOUSE/.

Fan Y, et al., 2015, Biotechnol Bioeng 112: 2172-2184.

Ghorbaniaghdam A, et al., 2014, PLoS ONE 9: e90832.

Higel F, et al., 2014, mAbs 6: 894-903.

Hossler P, et al., 2009, Glycobiology 19: 936-949.

Hu W-S, Zhou W, editors. 2012. Cell culture bioprocess engineering. Minnesota, Minn: University.

Hsu W-T, et al., 2012, Cytotechnology 64: 667-678.

Jayapal K P, et al., 2007, Chem Eng Prog 103: 40-47.

Konstantinidis S, et al., 2013, Biotechnol Bioeng 110: 1924-1935.

Leighty R W, Antoniewicz M R, 2011, Metab Eng 13: 745-755.

Lewis N E, et al., 2013, Nat Biotechnol 31: 759-765.

Maier K, et al., 2008, Biotechnol Bioeng 100: 355-370.

Waterston R H, et al., 2002, Nature 420: 520-562.

Müller C L, et al., 2009, Proceedings of the 11th Annual conference on Genetic and evolutionary computation. Montréal, Canada: ACM pp. 1411-1418. Available: http://dl.acm.org/citation.cfm?id=1570090.

Niklas J, et al., 2010, Curr Opin Biotechnol 21: 63-69.

Nolan R P, Lee K, 2011, Metab Eng 13: 108-124.

Nolan R P, Lee K, 2012, J Biotechnol 158: 24-33.

Oberhardt M A, et al., 2009, Mol Syst Biol 5, 320.

Ozturk S S, Palsson B O, 1990, Biotechnol Prog 6: 121-128.

Pais D A M, et al., 2014, Curr Opin Biotechnol 30C: 161-167.

Porter A J, et al., 2010, Biotechnol Prog 26: 1455-1464.

Porter A J, et al., 2010, Biotechnol Prog 26: 1446-1454.

Provost A, et al., 2006, Bioprocess Biosyst Eng 29: 349-366.

Rameez S, et al., 2014, Biotechnol Prog 30: 718-727.

Rathore A S, Winkle H, 2009, Nat Biotechnol 27: 26-34.

R Core Team. 2013. R: A language and environment for statistical computing. [Internet]. Vienna: R Foundation for Statistical Computing. Available: http://www.R-project.org.

Savinell J M, Palsson B O, 1992, J Theor Biol 154: 421-454.

Savinell J M, Palsson B O, 1992, J Theor Biol 154: 455-473.

Schaub J, et al., 2012, In: Hu W S, Zeng A-P, editors. Genomics and Systems Biology of Mammalian Cell Culture. Springer 617 Berlin Heidelberg, pp. 133-163

Selvarasu S, et al., 2010, Mol Biosyst 6: 152-161.

Selvarasu S, et al., 2010, J Biotechnol 150: 94-100.

Selvarasu S, et al., 2012, Biotechnol Bioeng 109: 1415-1429.

Sheikh K, et al., 2005, Biotechnol Prog 21: 112-121.

Stephanopoulos G, et al., 1998, Metabolic engineering: principles and methodologies. San Diego: Academic Press.

Tharmalingam T, et al., 2015, Biotechnol Bioeng 112: 1146-1154.

Vriezen N. 1998. Physiology of Mammalian Cells in Suspension Culture [Internet]. PhD Thesis, TU Delft. Available: http://repository.tudelft.nl/assets/uuid:2ca1b6f0-7894-4e63-8985-637 5b9b9eee1973/as_vriezen_19980526.PDF.

Wold S, et al., 2001, Chemom Intell Lab Syst 58: 109-130.

Zeck A, et al., 2012, PLoS ONE 7: e40328.

1. A method for determining if process data acquired during the cultivation of a mammalian or bacterial cell is affected by a problem comprising the following steps:
fitting the process data acquired during the cultivation of the mammalian or bacterial cell expressing a recombinant, heterologous polypeptide using a metabolic model generated for the same mammalian or bacterial cell expressing the same recombinant, heterologous polypeptide,
determining that the cultivation is affected by a problem if (i) the modeled fit shows an offset with respect to the raw data of more than 10%, or (ii) the modeled fit has a chi² value determined by a Pearson's chi-squared test of more than 5.

2. A method for selecting a cell expressing a heterologous polypeptide, wherein the method comprises the following steps:
a) separately cultivating a multitude of mammalian or bacterial cell clones that produce the same heterologous polypeptide, whereby during the cultivating temporal process data is recorded,
b) fitting the process data acquired in step a) of each clone individually using the same metabolic model, which had been generated for the same mammalian or bacterial cell,
c) determining that a cultivation of step a) is affected by a problem if the fit obtained in step b) for the process data of said cultivation obtained in step a) in said metabolic model (i) shows an offset with respect to the raw data of more than 10%, or (ii) the chi² value determined by a Pearson's chi-squared test for the fit is 5 or more,
d) repeating steps a) to c) with the clones that had a problem in the cultivation as determined in step c) or if no clone had a problem in the cultivation as determined in step c) selecting the clone from the multitude of clones as cell expressing a heterologous polypeptide that has (i) the highest titer, and/or (ii) the highest level of intended product quality attribute(s), and/or (iii) the preferred metabolic phenotype/highest rang in metabolic performance indicators.

3. The method according to any one of claims 1 to 2, wherein the chi² value determined by a Pearson's chi-squared test for the fit is 1 or more.

4. The method according to any one of claims 1 to 3, wherein the cell is (i) a mammalian cell is a CHO cell, and/or (ii) a bacterial cell is E. coli.

5. The method according to any one of claims 1 to 4, wherein the heterologous polypeptide is an antibody.

6. The method according to any one of claims 1 to 5, wherein the process data comprises the temporal values of at least 15 process parameters.

7. The method according to any one of claims 1 to 6, wherein the process data comprises the temporal values of at least 12 on-line process parameters and at least 28 off-line process parameters.

8. The method according to any one of claims 1 to 7, wherein the process data comprises at least 6 temporal values for each process parameter.

9. The method according to any one of claims 1 to 8, wherein
the metabolic model is a genome-based metabolic model, and/or
the metabolic model comprises the five compartments cytosol, mitochondria, endoplasmatic reticulum, Golgi apparatus and bioreactor, and/or
the metabolic model comprises the central metabolic pathways of glycolysis, citric acid cycle, pentose phosphate pathway, and respiratory chain, the biosynthesis of the major biomass constituents' protein, lipid, RNA, DNA, and carbohydrates, C1-metabolism, and amino acid degradation pathways.

10. The method according to any one of claims 1 to 9, wherein the metabolic model includes at least 600 reactions, 500 metabolites and 250 genes.

11. The method according to any one of claims 1 to 10, wherein the problem is a technical problem.
